SEQUENCE LISTING (19 sequences)

SEQ ID NO: 1-2 - skipped (2 entries)

SEQ ID NO: 3 - protein, 4 aa, synthetic construct
RRRR

SEQ ID NO: 4 - protein, 5 aa, synthetic construct
RRRRR

SEQ ID NO: 5 - protein, 6 aa, synthetic construct
RRRRRR

SEQ ID NO: 6 - skipped

SEQ ID NO: 7 - protein, 6 aa, synthetic construct
RGDRGD

SEQ ID NO: 8-13 - skipped (6 entries)

SEQ ID NO: 14 - protein, 4 aa, synthetic construct
GGGS

SEQ ID NO: 15 - protein, 4 aa, synthetic construct
GSGG

SEQ ID NO: 16 - protein, 5 aa, synthetic construct
GSGGG

SEQ ID NO: 17 - protein, 5 aa, synthetic construct
GSGGS

SEQ ID NO: 18 - protein, 5 aa, synthetic construct
GSGSG

SEQ ID NO: 19 - protein, 5 aa, synthetic construct
GGSGS

(19) United States
(12) Patent Application Publication
(10) Pub. No.: US 2023/0414759 A1
(43) Pub. Date: Dec. 28, 2023

(54) NOVEL CYCLIC PEPTIDES BASED ON NANOBIOSTRUCTURAL CONTROL, PEPTIDESOMES WITH CORE/SHELL STRUCTURE COMPRISING SAME, AND USES THEREOF

(71) Applicant: UIF (University Industry Fpimdatopm), Yonsei University, Seoul (KR)

(72) Inventors: Yong-Beom LIM, Seoul (KR); Hyoseok Kim, Seoul (KR)

(21) Appl. No.: 18/339,817

(22) Filed: Jun. 22, 2023

(30) Foreign Application Priority Data
Jun. 23, 2022 (KR) 10-2022-0076855

(51) Int. Cl. A61K 41/00; C07K 7/64; A61K 9/51; A61P 35/00

(52) U.S. Cl. CPC A61K 41/0057; A61P 35/00; A61K 9/5169; C07K 7/64

(57) ABSTRACT

The present disclosure relates to a novel cyclic peptide based on nano-biostructural control, a peptidesome with a core/shell structure including the same, and a use thereof. The cyclic peptide of the present disclosure may be prepared into a peptidesome having a vesicular structure consisting of a hollow core and a bilayer shell through self-assembly in a liquid. Since the prepared peptidesome is stable not only in vitro but also in vivo, especially against proteases in vivo, it can be usefully used as a drug carrier.

### CROSS-REFERENCE TO RELATED APPLICATION

This application claims priority to Korean Patent Application No. 10-2022-0076855 filed on Jun. 23, 2022, and all the benefits accruing therefrom under 35 U.S.C. § 119, the contents of which in its entirety are herein incorporated by reference.

### SEQUENCE LISTING

The instant application contains a Sequence Listing which has been submitted electronically in a computer readable Sequence Listing XML format and is hereby incorporated by reference in its entirety. Said computer readable Sequence Listing in XML format was created on Jun. 8, 2023, is named G1035-25401_SequenceListing.xml and is 11,511 bytes in size.

### BACKGROUND

### 1. Field

The present disclosure relates to a novel cyclic peptide based on nano-biostructural control, a peptidesome with a core/shell structure including the same, and uses thereof.

### 2. Description of the Related Art

Amino acids or peptides that constitute a protein tend to ‘self-assemble’ to form specific structures and shapes. Interests in self-assembled peptide nanostructures (SPNs) have been escalated in recent years. The SPNs have been utilized in various applications ranging from sensing and catalysts to therapeutics because they have very superior biocompatibility and various 2D and 3D structures can be fabricated easily by simply changing the amino acid sequences. Furthermore, the SPNs are advantageous since the nanostructural or functional diversity can be controlled through chemical modifications and the adoption of unique molecular topologies such as cyclic or dendritic structures in peptide supramolecular building blocks.

Vesicles are among the most widespread drug carrier applications of self-assembled nanostructures. As building blocks for self-assembly, lipids and synthetic polymers have been the most widely used than peptides. Most of the medical advancements in nanodrugs have been made with lipid building blocks. For example, Doxil, which is the first nanodrug approved by the FDA, is based on lipids. Lipids are also major components of exosomes or other extracellular vesicles and have recently drawn significant attention as potential drug carriers.

Although the vesicles such as liposomes, polymersomes and exosomes have been widely used as drug carriers, few researches have been made on the vesicles as described above. Peptides can play dual roles as a self-assembly building block and a bioactive functional unit. In order for peptide-based vesicles (hereinafter, also referred to as ‘peptidesomes’) to become successful drug delivery systems (DDSs), the issues related to differences in nanostructural properties between in vitro and in vivo conditions, which cause aggregation, cytotoxicity or decreased targeting ability, should be resolved. In particular, because peptides are sensitive to external environment, they may not work properly in vivo even when they have excellent effects in vitro.

The inventors of the present disclosure have prepared numerous self-assembled nanostructures and evaluated their functions in order to solve the problems described above. As a result, they found out that the following five issues have to be resolved for preparation of self-assembled nanostructures that can retain the same performance in vitro and in vivo: i) the selection of peptide building blocks and nanoscale size; ii) morphological transformation caused by drug loading; iii) inversely proportional relationship between intracellular delivery efficiency and cytotoxicity; iv) formation of a large aggregate under in-vivo environment; and v) maintenance of the structural stability of the SPN nanostructure in vivo.

The inventors of the present disclosure have developed a heuristic solution strategy to systemically resolve the problems occurring in peptidesomes, and thus developed a peptidesome with a new structure, which can exhibit the same effect in vitro and in vivo, and a cyclic peptide constituting the same.

As a result, the present disclosure proposes a cyclic peptide of a new structure, which has a nanoscale size, does not aggregate even after drug loading, exhibits low toxicity, has the capability of targeting cancer cells, is delivered directly into cells rather than through intracellular endosomes and has superior in-vivo stability with resistance to proteases, through the multivariate approach described above, and a peptidesome prepared therefrom.

### REFERENCES OF THE RELATED ART

### Patent Documents

(Patent document 1) Patent document 1. Korean Patent Registration No. 10-2353979.

### SUMMARY

The present disclosure is directed to providing a cyclic peptide capable of providing a peptidesome with superior drug delivery efficiency and stability.

The present disclosure is also directed to providing a peptidesome which can target cancer cells, provides superior drug efficacy because it is delivered directly into cells rather than through intracellular endosomes, and can exist stably in vivo with resistance to proteases.

The present disclosure is also directed to providing a pharmaceutical composition for preventing or treating cancer and a composition for diagnosing cancer, which contain the peptidesome.

The present disclosure provides a cyclic peptide including: (a) a hydrophilic peptide consisting of 2 to 12 L- or D-arginine residues; and (b) a hydrophobic peptide represented by General Formula 1, wherein the (a) and the (b) are linked by a linker.

Xaa1-Lys-Xaa2 [General Formula 1]

In General Formula 1, each of Xaa1 and Xaa2 is independently tryptophan (W) or phenylalanine (F).

In General Formula 1, Xaa1 may be bonded to the ε-amino group of the lysine residue (Lys).

The (a) may be a hydrophilic peptide consisting of 2 to 10 L- or D-arginine residues.

In the hydrophobic peptide (b), a hydrophobic ligand or a hydrophobic drug may be bonded to the α-amino group of the lysine residue.

The hydrophobic ligand may be any one selected from a C₈-C₂₄ fatty acid.

The fatty acid may be any one selected from a group consisting of oleic acid, lauric acid, palmitic acid, linoleic acid and stearic acid.

The hydrophobic drug may be any anticancer agent selected from doxorubicin, paclitaxel, cisplatin, sirolimus and etoposide or any photosensitizer selected from a phthalocyanine-based compound, a porphyrin-based compound, a fluorescein-based compound and a chlorin-based compound.

In General Formula 1, each of Xaa1 and Xaa2 may independently be tryptophan (W) or phenylalanine (F).

The linker may be any one selected from a linker peptide, Ebes and an oligoethylene glycol (OEG) represented by SEQ ID NOS 12-19.

The hydrophilic peptide (a) may have a sequence represented by any one selected from SEQ ID NOS 1-7.

The cyclic peptide may be any one selected from the compounds represented by Chemical Formulas 1-5.

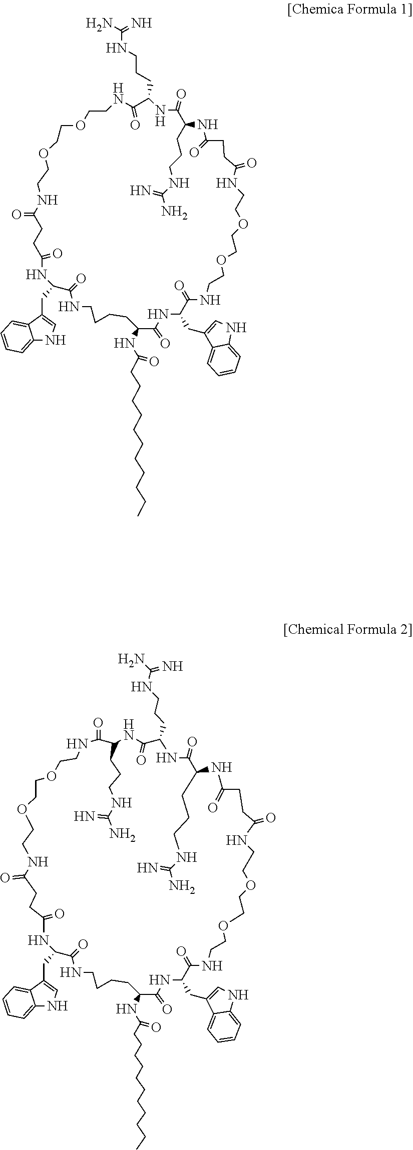

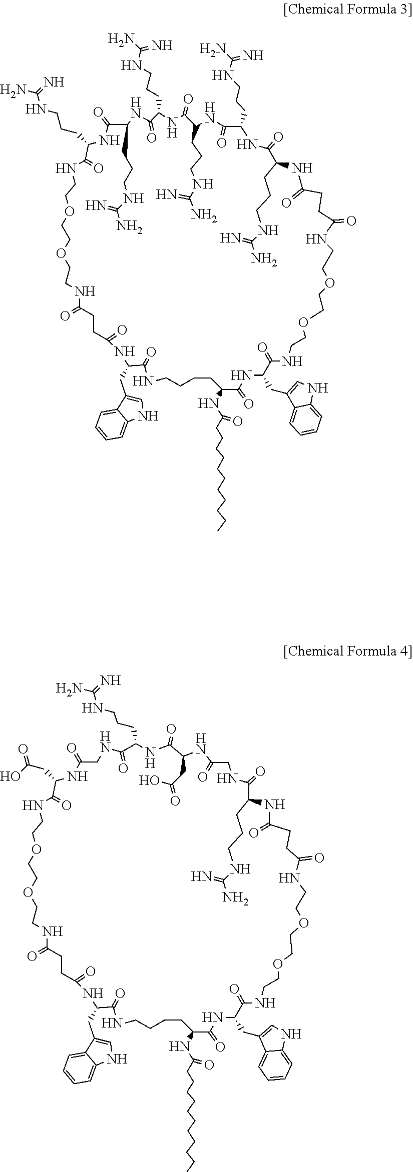

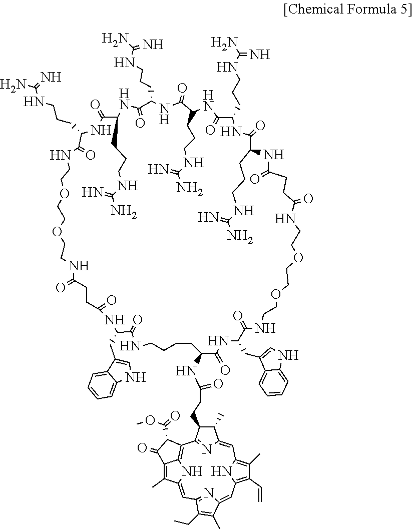

The cyclic peptide may self-assemble into a vesicular peptidesome in a solution.

The present disclosure also provides a spherical peptidesome having a vesicular structure, which is formed as at least one cyclic peptide according to claim 1 self-assembles in a liquid.

The peptidesome may consist of: a hollow core; and a shell having a bilayer structure, which includes the cyclic peptide.

A hydrophilic drug may be captured in the core moiety and a hydrophobic drug may be captured in the shell moiety so as to allow multiple drug release.

The peptidesome may have an average diameter of 10-150 nm.

The peptidesome may have an average shell thickness of 1-20 nm.

The cyclic peptide may be a mixture of two cyclic peptides having different hydrophilic peptides (a).

The mixture of cyclic peptides may be a mixture of a first cyclic peptide having a hydrophilic peptide selected from SEQ ID NOS 1-7 and a second cyclic peptide having a hydrophilic peptide selected from SEQ ID NOS 8-11.

The first cyclic peptide may be represented by any of Chemical Formulas 1-3 and the second cyclic peptide may be represented by Chemical Formula 4.

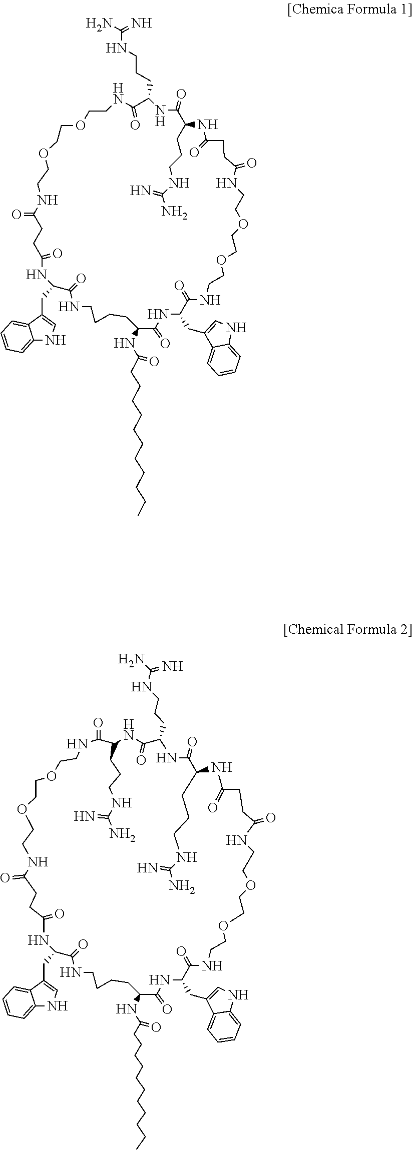

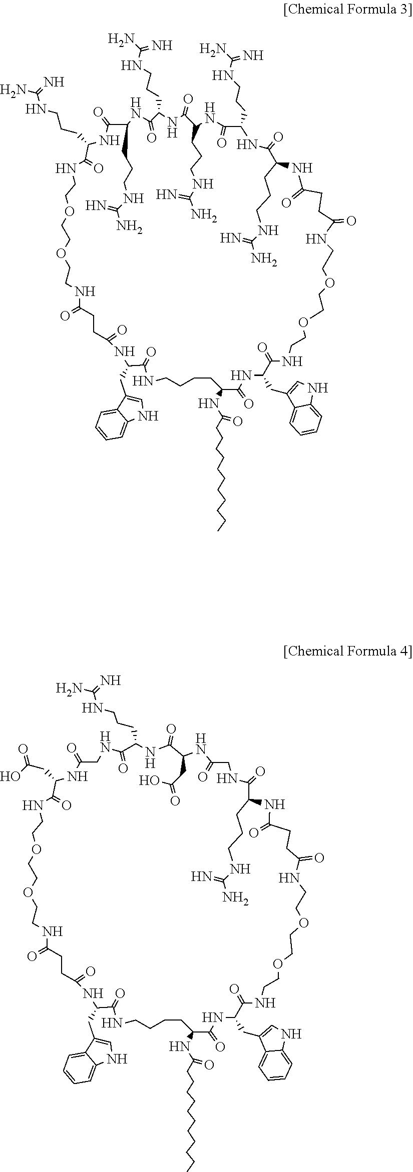

The mixture of cyclic peptides may include 1-50 mol % of the first cyclic peptide and the second cyclic peptide as the balance.

The liquid may be a solution containing one or more solution selected from a group consisting of glucose, a polyol and distilled water.

The liquid may be a solution containing 1-10 wt % of glucose, 10-30 wt % of a polyol and distilled water as the balance.

The polyol may be one or more polyol selected from a group consisting of ethylene glycol, propanediol, butanediol, pentanediol, hexanediol, glycerol and polyethylene glycol.

The present disclosure also provides a pharmaceutical composition for preventing or treating cancer, which contains the peptidesome and a drug encapsulated in the peptidesome.

The drug may be any one selected from a hydrophilic drug, a hydrophobic drug and a mixture thereof.

A hydrophilic drug may be encapsulated in a core of the peptidesome and a hydrophobic drug may be encapsulated in a shell having a bilayer structure of the peptidesome.

The hydrophobic drug may be any anticancer agent selected from doxorubicin, paclitaxel, cisplatin, sirolimus and etoposide or any photosensitizer selected from a phthalocyanine-based compound, a porphyrin-based compound, a fluorescein-based compound and a chlorin-based compound.

The peptidesome may penetrate directly into cancer cells rather than through endosomes and primarily release a hydrophobic drug, and then the peptidesome may be disrupted by photodynamically generated reactive oxygen species and secondarily release a hydrophilic drug contained in a core.

The cancer may be selected from a group consisting of lung cancer, stomach cancer, glioma, liver cancer, melanoma, kidney cancer, urothelial cancer, head and neck cancer, Merkel cell carcinoma, prostate cancer, blood cancer, breast cancer, mammary gland cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, nasopharyngeal cancer, thyroid cancer, bone cancer and a combination thereof.

The present disclosure also provides a composition for diagnosing cancer, which contains the peptidesome and a contrast agent.

According to the present disclosure, the cyclic peptide may form a peptidesome with a vesicular structure, having a hollow core and a bilayer shell, through self-assembly in a liquid.

The peptidesome of the present disclosure is stable both in vitro and in vivo. Especially, because it is stable against proteases in vivo, it can be usefully used as a drug carrier.

The peptidesome of the present disclosure can target and penetrate into cancer cells without an additional ligand. In addition, because a hydrophilic drug and a hydrophobic drug can be loaded in the core and shell, respectively, different drugs can be delivered with a single administration.

Because the cyclic peptide constituting the peptidesome of the present disclosure is a low-molecular-weight substance which is easy to synthesize, and the peptidesome is formed through self-assembly in a liquid, they can be prepared economically.

### BRIEF DESCRIPTION OF THE DRAWINGS

FIG. 1 schematically shows the general shapes of the existing nanodrug carriers.

FIG. 2a shows a result of purifying cyclic peptides prepared in Examples 2-1 to 2-5 and analyzing them with MALDI-TOF MS spectra. In FIG. 2a, R₂ indicates the cyclic peptide of Example 2-1, R₃ indicates the cyclic peptide of Example 2-2, R₆ indicates the cyclic peptide of Example 2-3, RGD₂ indicates the cyclic peptide of Example 2-4, and R₆-Pa indicates the cyclic peptide of Example 2-5.

FIG. 2b shows a result of purifying cyclic peptides prepared in Examples 2-1 to 2-5 and analyzing them with HPLC chromatograms. In FIG. 2B, R₂ indicates the cyclic peptide of Example 2-1, R₃ indicates the cyclic peptide of Example 2-2, R₆ indicates the cyclic peptide of Example 2-3, RGD₂ indicates the cyclic peptide of Example 2-4, and R₆-Pa indicates the cyclic peptide of Example 2-5.

FIG. 3 schematically shows the structure of a cyclic peptide prepared in Example 2-3 (R₆) according to the present disclosure.

FIG. 4 is an AFM image showing the self-assembly behavior of a cyclic peptide of Example 2-1 (R₂) in distilled water. The structure of a peptidesome prepared as the cyclic peptide (R₂) self-assembles into a vesicle in a liquid is shown at the top of FIG. 4.

FIG. 5A shows AFM images showing the self-assembly behavior of a cyclic peptide of Example 2-1 (R₂), FIG. 5B shows a cyclic peptide of Example 2-2 (R₃) and FIG. 5C shows a cyclic peptide of Example 2-3 (R₆) in distilled water.

FIG. 6 shows the structure and average diameter of peptidesomes, which are self-assembled nanostructures, in distilled water depending on the cone angle of cyclic peptides prepared in Examples 2-1 to 2-3 and temperature.

FIGS. 7A to 7C show results of measuring the average diameter of peptidesomes prepared from cyclic peptides of Example 2-1 (FIG. 7A), Example 2-2 (FIG. 7B) and Example 2-3 (FIG. 7C) depending on temperature (20° C., 30° C. and 40° C.) by DLS.

FIGS. 8A and 8B show the TEM images of a peptidesome (R₆) prepared from a cyclic peptide of Example 2-3. FIG. 8A is an enlarged image, and FIG. 8B shows a plurality of peptidesomes.

FIG. 9 shows the AFM images and structure of a peptidesome before encapsulation of a drug (R₆) and after encapsulation of a drug by sonication (R₆<-Pa).

FIG. 10 shows a result of analyzing the release of Pa (%) at 37° C. with time when a drug-encapsulated peptidesome of Example 4-3 (R₆<-Pa) was stored in PBS containing 2% (w/v) Tween 80.

FIG. 11 shows the UV absorption spectra of free Pa and a drug-encapsulated coassembled peptidesome of Example 4-6d (R₆:RGD₂<-Pa) (1:9).

FIG. 12 shows the AFM image of a peptidesome prepared from a cyclic peptide of Example 2-5 (R₆-Pa).

FIG. 13 shows a result of analyzing cell viability for a peptidesome of Example 3-3 (R₆), a peptidesome of Example 3-4 (RGD₂) and a coassembled peptidesome of Example 3-6 (R₆:RGD₂) at different concentrations.

FIG. 14 shows the fluorescence spectra of coassembled peptidesomes (R₆:RGD₂<-Pa) (Examples 4-6b to 4-6h) prepared varying the molar concentration of Pa. Excitation wavelength is 507 nm.

FIG. 15 shows a result of analyzing cell viability for a coassembled peptidesome of Example 4-6a (R₆:RGD₂<-Pa) (0.5:9.5), a coassembled peptidesome of Example 4-6d (R₆:RGD₂<-Pa) (1:9) and a coassembled peptidesome of Example 4-6i (R₆:RGD₂<-Pa) (1.5:8.5).

FIG. 16 and FIG. 17 are the TEM images of a coassembled peptidesome of Example 4-6d (R₆:RGD₂<-Pa) (1:9).

FIG. 18 is the AFM image of a coassembled peptidesome of Example 4-6d (R₆:RGD₂<-Pa) (1:9).

FIG. 19 schematically shows the structure of a coassembled peptidesome of Example 4-6d (R₆:RGD₂<-Pa) (1:9).

FIG. 20 shows a result of treating HeLa cells with a coassembled peptidesome of Example 4-6d (R₆:RGD₂<-Pa) (1:9) and then investigating fluorescence from Pa (red) by CLSM (confocal laser scanning microscopy).

FIGS. 21A and 21B show results of treating HeLa cells with a coassembled peptidesome of Example 4-6d (R₆:RGD₂<-Pa) (1:9) and then investigating fluorescence from Pa (red) and LysoTracker (green). A fluorescence image (FIG. 21A) and an enlarged image (FIG. 21B) are shown.

FIG. 22 shows a result of irradiating laser to SCC7 cells and analyzing the viability of the cells by MTT.

FIG. 23 shows a result of treating SCC7 cells with free Pa and a coassembled peptidesome of Example 4-6d (R₆:RGD₂<-Pa) (1:9) and measuring cell viability after laser irradiation for analysis of photodynamic anticancer efficacy.

FIG. 24 shows a result of quantifying singlet oxygen of free Pa and a coassembled peptidesome of Example 4-6d (R₆:RGD₂<-Pa) (1:9) with SOSG (Singlet Oxygen Sensor Green).

FIG. 25 shows an AFM image obtained after adding a coassembled peptidesome of Example 4-6d (R₆:RGD₂<-Pa) (1:9) to a serum-free RPMI 1640 medium.

FIGS. 26A and 26B show the AFM images of a coassembled peptidesome of Example 4-6d (R₆:RGD₂<-Pa) (1:9). FIG. 26A is an image obtained before NIR irradiation and FIG. 26B is an image obtained after NIR irradiation.

FIG. 27 shows the size distribution of a coassembled peptidesome of Example 4-6d (R₆:RGD₂<-Pa) (1:9) in the presence of serum proteins obtained by measuring average diameter.

FIGS. 28A to 28C show the CLSM images of SCC7 cells treated respectively with free Pa (FIG. 28A), a coassembled peptidesome of Example 4-6d (R₆:RGD₂<-Pa) (1:9) (DW) (FIG. 28B) and a coassembled peptidesome of Example 4-6d (R₆:RGD₂<-Pa) (1:9) (G&G) (FIG. 28C). In FIGS. 28A to 28C, red color indicates Pa and blue color indicates nucleus.

FIG. 29 shows a result of analyzing SCC7 cells treated respectively with free Pa, a coassembled peptidesome of Example 4-6d (R₆:RGD₂<-Pa) (1:9) (DW) and a coassembled peptidesome of Example 4-6d (R₆:RGD₂<-Pa) (1:9) (G&G) by flow cytometry (FACS).

FIG. 30 shows a result of analyzing the viability of SCC7 cells treated with free Pa, a coassembled peptidesome of Example 4-6d (R₆:RGD₂<-Pa) (1:9) (DW) and a coassembled peptidesome of Example 4-6d (R₆:RGD₂<-Pa) (1:9) (G&G), respectively. Error bars represent mean±standard deviation (n=3). Statistical significance was tested by two-sample Student's t-test and p<0.005 was regarded as significant (*** p<0.001).

FIG. 31 shows a result of analyzing ROS production from SSC7 cells treated with free Pa, a coassembled peptidesome of Example 4-6d (R₆:RGD₂<-Pa) (1:9) (DW) and a coassembled peptidesome of Example 4-6d (R₆:RGD₂<-Pa) (1:9) (G&G) by DCFDA. Error bars represent mean±standard deviation (n=3).

FIG. 32 shows the time-dependent whole-body NIR fluorescence images of a free Pa comparison group, a coassembled peptidesome of Example 4-6d (R₆:RGD₂<-Pa) (1:9) (DW) administration group and a coassembled peptidesome of Example 4-6d (R₆:RGD₂<-Pa) (1:9) (DW) administration group. The black dotted circles indicate tumor regions.

FIG. 32 shows the time-dependent whole-body NIR fluorescence images of a free Pa comparison group, a coassembled peptidesome of Example 4-6d (R₆:RGD₂<-Pa) (1:9) (DW) administration group and a coassembled peptidesome of Example 4-6d (R₆:RGD₂<-Pa) (1:9) (DW) administration group. The black dotted circles indicate tumor regions.

FIG. 33 shows the fluorescence microscopic images of organs isolated from a free Pa comparison group, a coassembled peptidesome of Example 4-6d (R₆:RGD₂<-Pa) (1:9) (DW) administration group and a coassembled peptidesome of Example 4-6d (R₆:RGD₂<-Pa) (1:9) (DW) administration group.

FIG. 34 shows fluorescence intensities quantified from the data of FIG. 33. Error bars represent mean±standard deviation (n=3).

FIG. 35 shows the fluorescence microscopic images of blood taken from a free Pa comparison group, a coassembled peptidesome of Example 4-6d (R₆:RGD₂<-Pa) (1:9) (DW) administration group and a coassembled peptidesome of Example 4-6d (R₆:RGD₂<-Pa) (1:9) (DW) administration group (top) and a quantification result thereof (bottom) (n=3).

FIG. 36 shows the fluorescence microscopic images of cryosection samples of cancer tissues taken from a free Pa comparison group, a coassembled peptidesome of Example 4-6d (R₆:RGD₂<-Pa) (1:9) (DW) administration group and a coassembled peptidesome of Example 4-6d (R₆:RGD₂<-Pa) (1:9) (DW) administration group.

FIG. 37 shows the images of cancer tissues isolated from a control group (saline), a free Pa comparison group and a coassembled peptidesome of Example 4-6d (R₆:RGD₂<-Pa) (1:9) (G&G) administration group after 14 days.

FIG. 38 shows a result of measuring the tumor size of a control group (saline), a free Pa comparison group and a coassembled peptidesome of Example 4-6d (R₆:RGD₂<-Pa) (1:9) (G&G) administration group at different times.

FIG. 39 shows a result of measuring the weight of cancer tissues isolated from a control group (saline), a free Pa comparison group and a coassembled peptidesome of Example 4-6d (R₆:RGD₂<-Pa) (1:9) (G&G) administration group after 14 days.

FIG. 40 shows the fluorescence microscopic images of cancer tissues isolated from a control group (saline), a free Pa comparison group and a coassembled peptidesome of Example 4-6d (R₆:RGD₂<-Pa) (1:9) (G&G) administration group and stained with H&E after 14 days.

FIG. 41 shows a result of measuring the change in body weight of a control group (saline), a free Pa comparison group and a coassembled peptidesome of Example 4-6d (R₆:RGD₂<-Pa) (1:9) (G&G) administration group with time.

FIG. 42 shows the fluorescence microscopic images of major organs (heart, lung, liver, spleen and kidney) isolated from a control group (saline), a free Pa comparison group and a coassembled peptidesome of Example 4-6d (R₆:RGD₂<-Pa) (1:9) (G&G) administration group and stained with H&E after 14 days.

FIG. 43 shows an HPLC analysis result of a peptidesome of Example 3-4 (RGD₂) in the presence of the protease trypsin.

### DETAILED DESCRIPTION

Hereinafter, the present disclosure is described in detail.

In general, supramolecular building blocks including lipids, polymers and peptides assemble into common morphologies such as spherical micelles, cylindrical micelles and vesicles. Among them, vesicles have been the most widely used as drug carriers. Intracellular transport systems, exosomes, enveloped viruses, etc. are also vesicles having self-assembled morphologies. One of the biggest advantageous features of the vesicles is that different drugs can be loaded.

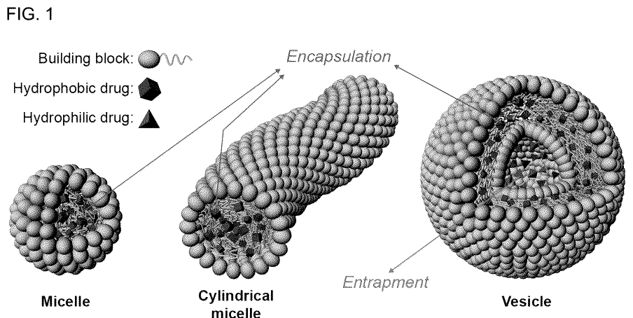

FIG. 1 schematically show the morphologies of the three generally used self-assembled nanostructures in which low-molecular-weight drugs are loaded. Nonpolar drugs may be incorporated in the hydrophobic space of the shell of the vesicles via an encapsulation mechanism, and polar drugs may be incorporated in the core of the vesicles via an entrapment mechanism. In contrast, the spherical and cylindrical micelles are disadvantageous in that they can capture only one drug. Therefore, the inventors have designed new peptides having both the usefulness of vesicles and the multifunctionality of peptides as supramolecular building blocks. The present disclosure presents peptidesomes, which are new type of self-assembled nanostructures consisting only of the newly designed peptides, which can be utilized as drug carriers, etc. having superior intracellular delivery efficacy and drug encapsulation efficiency.

In an aspect, the present disclosure relates to a cyclic peptide including: (a) a hydrophilic peptide consisting of 2 to 12 L- or D-arginine residues; and (b) a hydrophobic peptide represented by General Formula 1, wherein the (a) and the (b) are linked by a linker.

Xaa1-Lys-Xaa2 [General Formula 1]

In General Formula 1, each of Xaa1 and Xaa2 is independently tryptophan (W) or phenylalanine (F).

The cyclic peptide according to the present disclosure can self-assemble to form a peptidesome having superior drug delivery efficacy, encapsulation efficiency and structural stability and has the following advantages.

1) Since the cyclic peptide according to the present disclosure has a cyclic, not linear, structure, it has a strong tendency to self-assemble into a peptidesome having a vesicular structure. Accordingly, the yield of the peptidesome is high when the same amount of the peptide is dispersed in a liquid. Therefore, it can be utilized as a kit for a drug carrier which is stored in a separate container until use.

2) The cyclic peptide according to the present disclosure can ensure effective intracellular potential since it contains 2 to 12 arginine residues that play a major role in cell surface targeting and membrane potential.

3) The cyclic peptide according to the present disclosure may self-assemble in vivo to form a peptidesome with superior intracellular delivery efficiency, with a size of smaller than 200 nm, specifically smaller than 150 nm, more specifically smaller than 100 nm.

4) The cyclic peptide according to the present disclosure can be synthesized easily with high yield because it has a simple structure and a small molecular weight.

The (a) may be a hydrophilic peptide consisting of 2 to 10 L- or D-arginine residues, more specifically a hydrophilic peptide consisting of 2 to 6 L- or D-arginine residues, further more specifically a hydrophilic peptide consisting of 3 to 6 L- or D-arginine residues, most specifically a hydrophilic peptide consisting of 2 or 6 L- or D-arginine residues. In this case, a peptidesome having an average diameter smaller than 100 nm may be formed through self-assembly under the temperature condition of 20-40° C.

In the cyclic peptide according to the present disclosure, the hydrophilic peptide (a) may have a sequence represented by any one of SEQ ID NOS 1-7, specifically by any one of SEQ ID NOS 1, 2, 5 and 7.

[SEQ ID NO 1]
RR
[SEQ ID NO 2]
RRR
[SEQ ID NO 3]
RRRR
[SEQ ID NO 4]
RRRRR
[SEQ ID NO 5]
RRRRRR
[SEQ ID NO 6]
RGD
[SEQ ID NO 7]
RGDRGD

In General Formula 1, Xaa1 may be bonded to the ε-amino group of the lysine residue (Lys). When Xaa1 is bonded to the ε-amino group of the lysine residue (Lys), it is more advantageous for stabilizing the cyclic structure of the cyclic peptide of the present disclosure.

In General Formula 1, each of Xaa1 and Xaa2 may be specifically tryptophan (W) or phenylalanine (F), more specifically tryptophan (W).

The hydrophobic peptide represented by General Formula 1 may be represented by any of SEQ ID NOS 8-11, specifically by any of SEQ IDS NO 7-10, most specifically by SEQ ID NO 10.

[SEQ ID NO 8]
WKQ
[SEQ ID NO 9]
QKW
[SEQ ID NO 10]
WKW
[SEQ ID NO 11]
QKQ

The linker is not specially limited as long as it is a linker having a flexible structure for peptide bonding and widely known in the art. For example, it may be any one selected from a linker peptide, Ebes and an oligoethylene glycol (OEG) represented by SEQ ID NOS 12-19, most specifically Ebes.

[SEQ ID NO 12]
GS
[SEQ ID NO 13]
GSG
[SEQ ID NO 14]
GGGS
[SEQ ID NO 15]
GSGG
[SEQ ID NO 16]
GSGGG
[SEQ ID NO 17]
GSGGS
[SEQ ID NO 18]
GSGSG
[SEQ ID NO 19]
GGSGS

The Ebes may be represented by Structural Formula a.

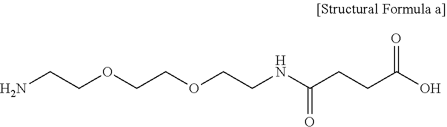 [SMILES: NCCOCCOCCNC(=O)CCC(=O)O]

In the hydrophobic peptide (b), a hydrophobic ligand or a hydrophobic drug may be bonded to the α-amino group of the lysine residue. When a hydrophobic ligand is bonded, it may act as a drug carrier after a peptidesome is formed. When a hydrophobic drug is bonded, it may act both as a drug carrier and a prodrug. But, when a hydrophobic drug is bonded, a peapod-like nanostructure is formed. Accordingly, it is preferred that a hydrophobic ligand is bonded to the α-amino group of the lysine residue of the hydrophobic peptide (b).

The hydrophobic ligand may be any one selected from a C₈-C₂₄ fatty acid. The C₈-C₂₄ fatty acid may specifically be a C₁₂-C₂₀ saturated or unsaturated fatty acid. The fatty acid may be more specifically any one selected from a group consisting of oleic acid, lauric acid, palmitic acid, linoleic acid and stearic acid, most specifically oleic acid or lauric acid, although not being limited thereto.

The hydrophobic drug may be any anticancer agent selected from doxorubicin, paclitaxel, cisplatin, sirolimus and etoposide or any photosensitizer selected from a phthalocyanine-based compound, a porphyrin-based compound, a fluorescein-based compound and a chlorin-based compound.

The photosensitizer may be any one selected from a group consisting of a phthalocyanine-based compound, a porphyrin-based compound, a fluorescein-based compound and a chlorin-based compound, specifically any one selected from a group consisting of phthalocyanine, zinc phthalocyanine, copper phthalocyanine, Photofrin, Photogem, Radachlorin, chlorin e6, pheophorbide A and rose bengal. More specifically, pheophorbide A or rose Bengal may be used as the photosensitizer because cancer cells in deep tissues can be damaged or killed more effectively and a synergistic effect may be achieved in photodynamic therapy.

The cyclic peptide may be any one selected from compounds represented by Chemical Formulas 1-5, specifically any one selected from a compound represented by Chemical Formula 3 or 4.

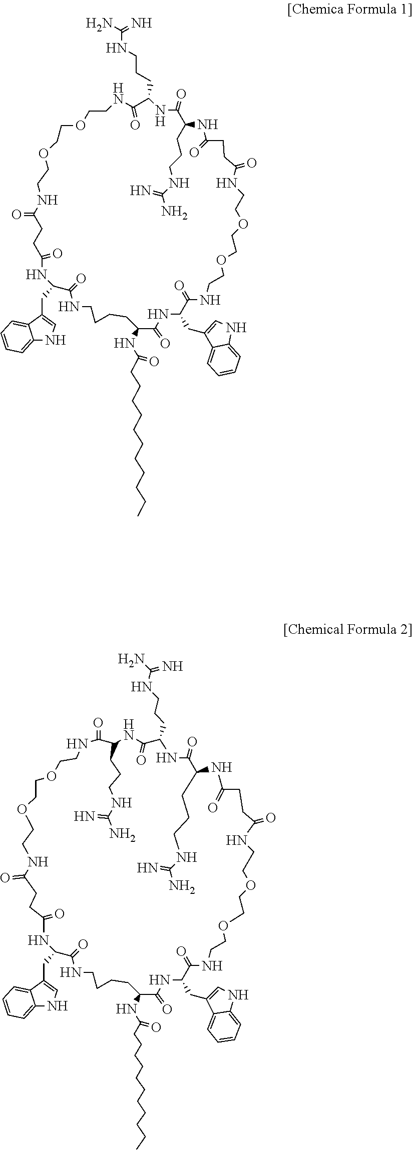

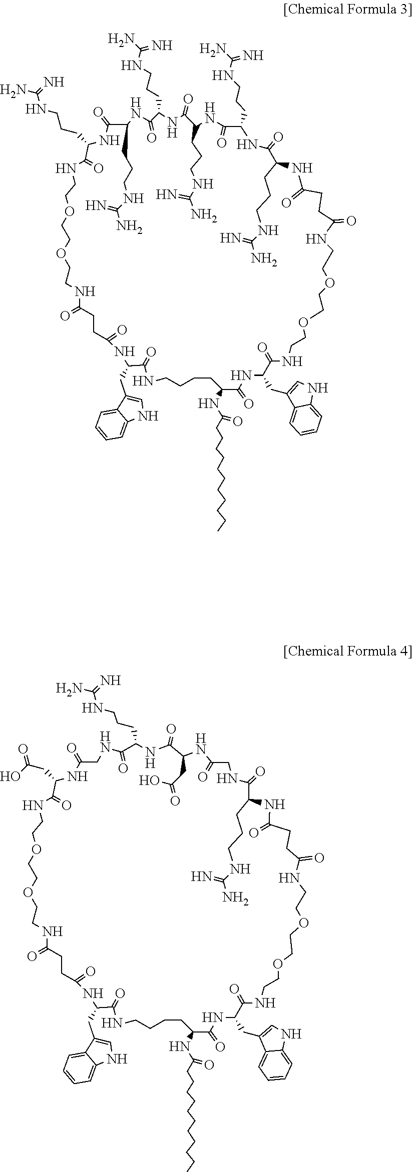

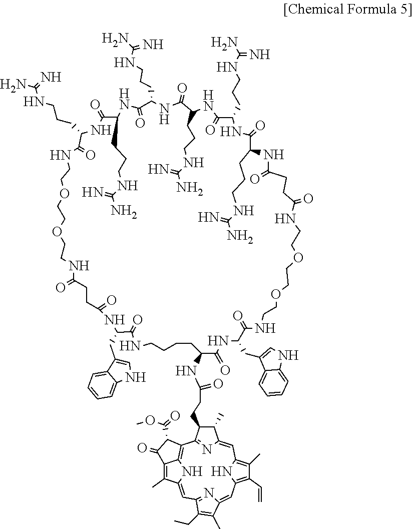

The cyclic peptide may self-assemble into a vesicular peptidesome in a solution.

In another aspect, the present disclosure relates to a spherical peptidesome having a vesicular structure, which is formed as at least one of the above-described cyclic peptides self-assembles in an aqueous solution.

The peptidesome may consist of: a hollow core; and a shell having a bilayer structure, which includes the cyclic peptide.

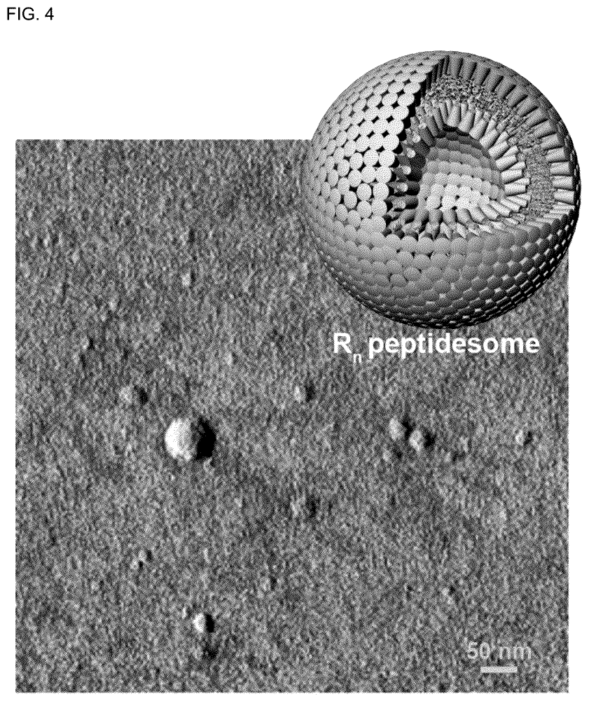

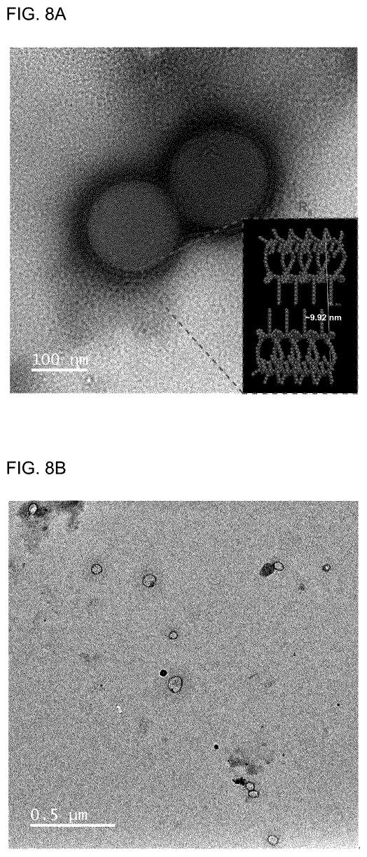

A hydrophilic drug may be captured in the core moiety of the peptidesome and a hydrophobic drug may be captured in the shell moiety so as to allow multiple drug release. The bilayer shell may form a structure in which a plurality of the cyclic peptides are lined up side by side to form a layer and the two cyclic peptide layers are gathered to form a bilayer (see FIG. 4 and FIG. 8A). The cyclic peptide may have a bilayer structure like that of a phospholipid bilayer because the polar ring head (hydrophilic peptide moiety) has hydrophilicity and the nonpolar ring bottom and tail (hydrophobic peptide and/or hydrophobic ligand (or hydrophobic drug) moiety) have hydrophobicity.

The bilayer shell is a bilayer structure formed as the two cyclic peptide layers are gathered. The ring head of the cyclic peptide is directed toward the outside of the shell and the ring bottom and tail are arranged to face toward the inside of the shell. The bilayer structure may be maintained firmly through the interaction between the arranged cyclic peptides. Whereas general phospholipid bilayers are degraded easily in vivo due to the fluidity of the hydrophobic structure, the peptidesome according to the present disclosure may have a stable structure both in vivo and ex vivo through molecular arrangement because it contains the cyclic peptide designed through the above-described process.

The peptidesome may have an average diameter of 10-150 nm. Specifically, the peptidesome may have an average diameter of 50-130 nm and a nanoscale size of 100 nm depending on temperature. Specifically, it has an average diameter of 50-90 nm at 20-30° C. That is to say, the average diameter is decreased at low temperature. It can be seen that the peptidesome is very easy to store at low temperature because the structure of the peptidesome becomes tighter and more robust.

The peptidesome may have an average shell thickness of 1-20 nm, specifically 1-15 nm, 5-15 nm, 8-13 nm, 9-12 nm or 9-11 nm.

The cyclic peptide may be a mixture of two cyclic peptides having different hydrophilic peptides (a). Use of two cyclic peptides provides the advantage that functions can be complemented without structural and morphological changes as compared to when only one cyclic peptide is used. Since all the cyclic peptides according to the present disclosure are amphiphilic peptides and have the same conical structure, peptidesomes can be formed easily even when they are mixed. However, since the two-dimensional structure may be changed when a sequence different from the hydrophilic peptide according to the present disclosure is added, deleted or substituted, it is preferred that the sequences of the cyclic peptides are selected from the sequences according to the present disclosure.

Specifically, the mixture of the cyclic peptides may be a mixture of a first cyclic peptide having a hydrophilic peptide (a) selected from SEQ ID NOS 1-7 and a second cyclic peptide having a hydrophilic peptide (b) selected from SEQ ID NOS 8-11 in order to improve function only without negative effects on the overall shape or structure of the peptidesome according to the present disclosure as a drug carrier.

More specifically, the first cyclic peptide may be represented by any one of Chemical Formulas 1-3 and the second cyclic peptide may be represented by Chemical Formula 4. Most specifically, a mixture of a first cyclic peptide represented by Chemical Formula 3 and a second cyclic peptide represented by Chemical Formula 4 may be used.

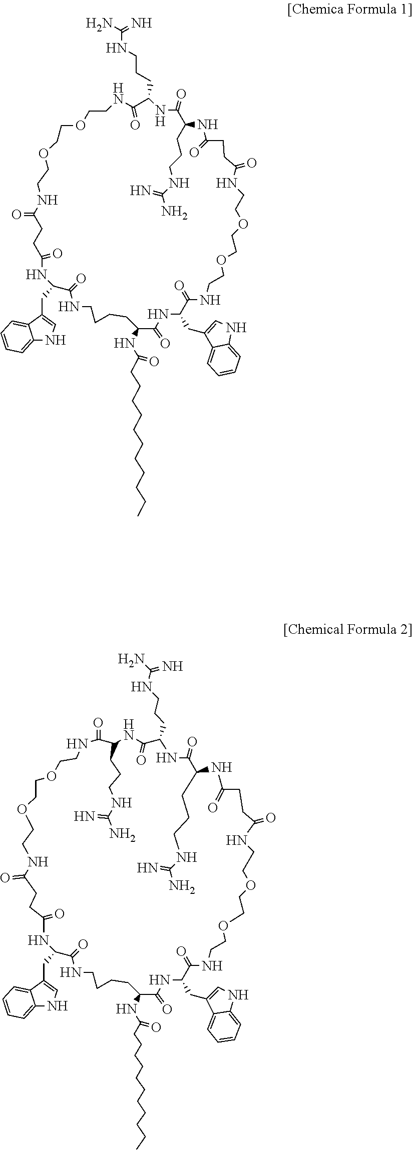

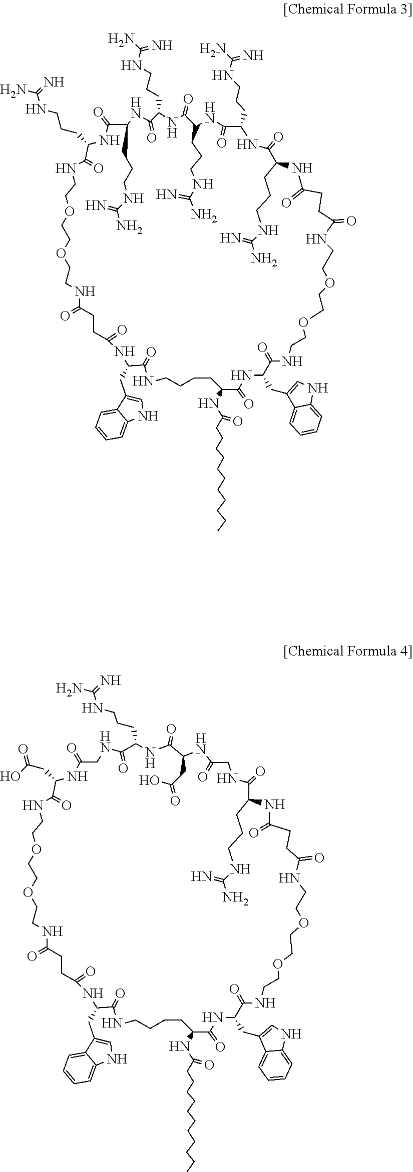

For remarkably superior intracellular delivery efficiency, stability, biocompatibility, etc., the mixture of the cyclic peptides may be a mixture of the first cyclic peptide and the second cyclic peptide at an appropriate ratio. The mixture of the cyclic peptides may be specifically a mixture of 1-50 mol % of the first cyclic peptide and the second cyclic peptide as the balance, more specifically 1-50 mol % of the first cyclic peptide and 50-99 mol % of the second cyclic peptide, further more specifically 1-30 mol % of the first cyclic peptide and the second cyclic peptide as the balance, further more specifically 1-30 mol % of the first cyclic peptide and 70-99 mol % of the second cyclic peptide, most specifically 5-15 mol % of the first cyclic peptide and the second cyclic peptide as the balance or 85-95 mol % of the second cyclic peptide. When the mixing ratio is satisfied, intracellular delivery efficiency is superior and in-vivo stability is the most superior with no toxicity at all.

The liquid in which the peptidesome of the present disclosure is self-assembled is not specially limited as long as it is a solution in which the cyclic peptide according to the present disclosure can self-assemble to form a peptidesome. Specifically, a solution containing one or more selected from a group consisting of glucose, a polyol and distilled water may be used. Specifically, the peptidesome according to the present disclosure maintains the average diameter of 150 nm regardless of the solution, which does not cause a problem in performing various functions. In particular, since the particle size does not have a significant effect on intracellular delivery efficiency, there is no particular limitation as long as the average diameter is 150 nm. However, when the average diameter needs to be maintained smaller than 110 nm according to the purpose of use of the peptidesome, it is preferred to use an aqueous glucose solution or an aqueous polyol solution. Glucose is a biocompatible molecule and is a stable substance widely used in injectable preparations.

In an example that will be described below, it was confirmed that the peptidesome has an average diameter of 100 nm when it is self-assembled or stored in a solution containing glucose, a polyol and distilled water. Accordingly, a solution containing glucose, a polyol and distilled water is the most preferred for structural stabilization of the peptidesome according to the present disclosure.

It is thought that the polyol increases the colloidal stability of the peptidesome according to the present disclosure and maintains in-vivo osmolarity and tonicity after injection of the peptidesome. It was confirmed that the peptidesome according to the present disclosure has a size of smaller than 150 nm, smaller than 140 nm, smaller than 130 nm or smaller than 120 nm without forming an aggregate in a solution containing one or more selected from a group consisting of glucose, a polyol and distilled water. More specifically, a mixture of glucose, a polyol and distilled water, further more specifically a mixture containing 1-10 wt % glucose, 10-30 wt % of a polyol and distilled water as the balance, further more specifically a mixture containing 2-7 wt % of glucose, 15-25 wt % of a polyol and distilled water as the balance, most specifically a mixture containing 4-6 wt % of glucose, 17-22 wt % of a polyol and distilled water as the balance, may be used. When the above ranges are satisfied, the average diameter of the peptidesome may be maintained smaller than 100 nm.

The polyol is not specially limited as long as it has superior biocompatibility. It may be specifically one or more polyol selected from a group consisting of ethylene glycol, propanediol, butanediol, pentanediol, hexanediol, glycerol and polyethylene glycol, more specifically one or more polyol selected from a group consisting of ethylene glycol, glycerol and polyethylene glycol, most specifically glycerol.

When the peptidesome according to the present disclosure is stored in a mixture of 5 wt % of glucose, 20 wt % of glycerol and distilled water as the balance (hereinafter, also referred to as ‘G&G’), aggregation can be prevented and an average diameter of smaller than 100 nm can be maintained.

The peptidesome having the structure described above can be delivered into a target cell in the body to deliver a drug directly into the target cell. The target cell may be a disease-related cell such as a cancer cell, an inflammatory cell, etc. Specifically, it may be a cancer cell.

The cancer cell may be a cell associated with one or more tumor disease selected from a group consisting of neoplasia, mantle cell lymphoma, multiple myeloma (e.g., metastatic multiple myeloma), lung cancer, non-small-cell lung cancer (e.g., metastatic non-small-cell lung cancer or non-small-cell lung carcinoma), small-cell lung carcinoma, solid tumor, lymphoma (e.g., lymphoplasmacytic lymphoma, diffuse large B-cell lymphoma, non-Hodgkin lymphoma, follicular lymphoma or peripheral T-cell lymphoma), chronic lymphocytic leukemia, T-cell prolymphocytic leukemia, breast cancer (e.g., metastatic breast cancer), cervical cancer, colorectal cancer, colon cancer, melanoma, prostate cancer (e.g., hormone-refractory prostate cancer), pancreatic cancer (e.g., metastatic pancreatic cancer), ovarian cancer, glioblastoma (e.g., glioblastoma multifome), head squamous cell carcinoma, neck squamous cell carcinoma, amyloidosis (e.g., primary systemic amyloidosis), bone disease, blood cancer, graft-versus-host disease, Waldenström macroglobulinemia, smoldering myeloma and monoclonal gammopathy of undetermined significance (MGUS).

Because the peptidesome according to the present disclosure is delivered directly into cancer cells rather than via endosomes, it can retain or enhance the efficacy of the drug.

The drug may be one or more selected from a group consisting of a hydrophilic drug, a hydrophobic drug and a mixture thereof. A hydrophilic drug may be encapsulated in the core of the peptidesome, and a hydrophobic drug may be encapsulated in the shell having a bilayer structure of the peptidesome.

The hydrophilic drug is not specially limited as long as it is a drug exhibiting hydrophilicity and can be encapsulated in the core of the peptidesome according to the present disclosure. For example, it may be an ion, a low-molecular-weight drug, a gene drug, a protein drug or a mixture thereof, and may include a contrast agent for diagnosis or detection of cancer cells.

The low-molecular-weight drug is not specially limited as long as it is a low-molecular-weight substance exhibiting hydrophilicity. For example, it may be antipyrin, antifebrin, aspirin, salipyrin, salicylate, ibuprofen, flurbiprofen, piroxicam, naproxen, fenoprofen, indomethacin, phenylbutazone, methotrexate, mechlorethamine, dexamethasone, prednisolone, celecoxib, valdecoxib, nimesulide, cortisone or corticosteroid, although not being limited thereto.

The gene drug may be a small interfering RNA (siRNA), a small hairpin RNA (shRNA), a microRNA (miRNA) or a plasmid DNA, although not being limited thereto.

The protein drug may be a monoclonal antibody-based drug such as trastuzumab, rituximab, bevacizumab, cetuximab, bortezomib, erlotinib, gefitinib, imatinib mesylate and sunitinib, an enzyme such as L-asparaginase, or a hormone-based drug such as triptorelin acetate, megestrol acetate, flutamide, bicalutamide, goserelin, cytochrome c or p53 protein, although not being limited thereto.

The hydrophobic drug may be any anticancer agent selected from doxorubicin, paclitaxel, cisplatin, sirolimus and etoposide or any photosensitizer selected from a phthalocyanine-based compound, a porphyrin-based compound, a fluorescein-based compound and a chlorin-based compound.

The photosensitizer may be any one selected from a phthalocyanine-based compound, a porphyrin-based compound, a fluorescein-based compound and a chlorin-based compound, specifically any one selected from phthalocyanine, zinc phthalocyanine, copper phthalocyanine, Photofrin, Photogem, Radachlorin, chlorin e6, pheophorbide A and rose bengal. More specifically, pheophorbide A or rose Bengal may be used as the photosensitizer because cancer cells in deep tissues can be damaged or killed more effectively and a synergistic effect may be achieved in photodynamic therapy.

In another aspect, the present disclosure relates to a composition for preventing or treating cancer, which contains a peptidesome and a drug encapsulated in the peptidesome.

In the present disclosure, the drug encapsulated in the peptidesome may be mixed in an amount of less than 200 molar parts, specifically 1-100 molar parts, more specifically 1-50 molar parts, most specifically 1-25 molar parts, based on 100 molar parts of the peptidesome. An amount of less than 200 molar parts is preferred because crystallinity may be insufficient if the amount of the drug exceeds 200 molar parts. If the amount exceeds 25 molar parts, although a nanostructure encapsulating the drug is formed through self-assembly, it is extended and elongated to form a peapod-like nanostructure. Therefore, it is the most preferred that the drug is contained in an amount less than 25 molar parts in order to obtain a peptidesome with a spherical vesicular structure through self-assembly. The lower limit may be 1 molar part or more, but there is no problem even when it is contained in an amount of 0 molar part (meaning that the drug is not contained).

The drug may be one or more selected from a group consisting of a hydrophilic drug, a hydrophobic drug and a mixture thereof. A hydrophilic drug is encapsulated in the core of the peptidesome and a hydrophobic drug is encapsulated in the shell having a bilayer structure of the peptidesome.

The hydrophobic drug may be any anticancer agent selected from doxorubicin, paclitaxel, cisplatin, sirolimus and etoposide or any photosensitizer selected from a phthalocyanine-based compound, a porphyrin-based compound, a fluorescein-based compound and a chlorin-based compound. Further details may be referred to the above description of the ‘peptidesome’.

The description of the hydrophilic drug may be referred to the above description of the ‘peptidesome’.

The peptidesome may penetrate directly into cancer cells rather than through endosomes and primarily release a hydrophobic drug, and then the peptidesome may be disrupted by photodynamically generated reactive oxygen species and secondarily release a hydrophilic drug contained in the core.

In the present disclosure, the ‘cancer cell’ refers to a cancer tissue or a benign or malignant type of cell, and may be used interchangeable with the term tumor cell.

In the present disclosure, the ‘cancer’, also called malignant tumor or neoplasm, refers to a disease related with cell death. It refers to a condition or disease caused by excessive proliferation of cells as a result of the breakdown of the normal balance of cell death. In some cases, the abnormally hyperproliferating cells invade nearby tissues and organs to form lumps and destroy or transform the normal structures in the body.

The cancer may be solid cancer or blood cancer, and may also be primary or metastatic. The cancer may be selected from a group consisting of lung cancer, stomach cancer, glioma, liver cancer, melanoma, kidney cancer, urothelial cancer, head and neck cancer, Merkel cell carcinoma, prostate cancer, blood cancer, breast cancer, mammary gland cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, nasopharyngeal cancer, thyroid cancer, bone cancer and combination thereof, although not being limited thereto. The cancer may be caused by mutation of specific genes.

In the present disclosure, prevention refers to any action of suppressing or delaying the onset of a disease by administering the pharmaceutical composition of the present disclosure, and treatment refers to any action of ameliorating or favorably changing the symptoms of a disease that has already occurred by administering the pharmaceutical composition of the present disclosure.

In the present disclosure, the term ‘treatment or prevention’ includes alleviating the symptoms of a disease, condition or disorder by preventing or delaying the onset of the symptoms, complications or biochemical signs of a disease (e.g., cancerous disease or disorder), or preventing or inhibiting further occurrence of a disease, condition or disorder. The treatment may refer to prophylactic suppression of symptoms after a disease has occurred (for preventing or delaying the development of the disease or preventing the development of clinical or subclinical symptoms thereof) or therapeutic suppression or alleviation.

In the present disclosure, the pharmaceutical composition may further contain an adequate carrier, excipient or diluent according to conventional methods. Examples of the carrier, excipient and diluent that may be contained in the pharmaceutical composition of the present disclosure include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, methyl hydroxybenzoate, propyl hydroxybenzoate, talc, magnesium stearate, mineral oil, etc., although not being limited thereto.

The pharmaceutical composition according to the present disclosure may be formulated into an oral formulation such as a powder, a granule, a tablet, a capsule, a suspension, an emulsion, a syrup, an aerosol, etc., a formulation for external application, a suppository or a sterilized injection solution according to common methods.

Specifically, they may be prepared using a commonly used diluent or excipient such as a filler, an extender, a binder, a wetting agent, a disintegrant, a surfactant, etc. Solid formulations for oral administration include a tablet, a pill, a powder, a granule, a capsule, etc., and these solid formulations may be prepared by mixing the pharmaceutical composition of the present disclosure with at least one excipient, e.g., starch, calcium carbonate, sucrose, lactose, gelatin, etc. Furthermore, lubricants such as magnesium stearate or talc may also be used in addition to the simple excipients. Liquid formulations for oral administration include a suspension, a liquid for internal use, an emulsion, a syrup, etc. They may contain, in addition to a commonly used simple diluent such as water or liquid paraffin, various excipients such as a wetting agent, a sweetener, an aromatic, a preservative, etc. Formulations for parenteral administration include a sterilized aqueous solution, a nonaqueous solution, a suspension, an emulsion, a freeze-dried formulation and a suppository. For the nonaqueous solution or suspension, propylene glycol, polyethylene glycol, vegetable oil such as olive oil, an injectable ester such as ethyl oleate, etc. may be used. As a base for the suppository, witepsol, macrogol, Tween 61, cocoa butter, laurin butter, glycerogelatin, etc. may be used.

The pharmaceutical composition of the present disclosure may be administered to a mammal such as rat, mouse, livestock, human, etc. via various routes.

The pharmaceutical composition of the present disclosure may be in any form suitable for the intended administration method. The administration of the pharmaceutical composition of the present disclosure refers to the introduction of the composition to a patient by any suitable method, and the pharmaceutical composition may be administered through any general route as long as the drug can reach the target tissue.

The administration route of the pharmaceutical composition according to the present disclosure may be an oral or parenteral administration route, although not being limited thereto. The parenteral administration route may include buccal, intravenous, intramuscular, intraarterial, intramedullary, intraarticular, intrasynovial, intrasternal, intrathecal, intracardiac, transdermal, subcutaneous, intradermal, intraperitoneal, intranasal, intestinal, topical, intracranial, intracerebroventricular, intrauterine, sublingual or rectal routes. The pharmaceutical composition of the present disclosure may be administered by any device capable of delivering the active ingredient to a target site.

The content of the active ingredient in the pharmaceutical composition may be adjusted adequately depending on the purpose use, formulation type, etc. of the pharmaceutical composition and may be, for example, 0.001-99 wt %, 0.001-90 wt %, 0.001-50 wt %, 0.01-50 wt %, 0.1-50 wt % or 1-50 wt % based on the total weight of the pharmaceutical composition, although not being limited thereto

The administration dose of the pharmaceutical composition of the present disclosure may vary depending on various factors including the activity of the active ingredient, age, body weight, general health, sex, diet, administration route, excretion rate, drug combination and the severity of a specific disease to be prevented or treated.

The administration dose of the pharmaceutical composition may be appropriately selected by those skilled in the art although it may vary depending on the patient's condition and body weight, the severity of a disease, drug form, and administration route and period. 0.0001-50 mg/kg or 0.001-50 mg/kg may be administered per day. The pharmaceutical composition may be administered once a day or may be administered in several divided doses. The administration dose does not limited the scope of the present disclosure in any way. The pharmaceutical composition according to the present disclosure may be formulated as a pill, a sugar-coated tablet, a capsule, a liquid, a gel, a syrup, a slurry or a suspension.

In another aspect, the present disclosure relates to a composition for detecting and diagnosing cancer, which contains the peptidesome and a contrast agent.

Since the peptidesome has cancer cell-targeting ability and has excellent intracellular delivery efficiency, it exists stably outside the target cell and accurately delivers the contrast agent into the target cell, enabling accurate detection and diagnosis of cancer cells. For example, since it penetrates into a cancer tissue, particularly into cancer cells, and releases the contrast agent, it can be used for diagnosis of various types of cancer.

The contrast agent may include one or more selected from a group consisting of a paramagnetic material as an MRI contrast agent, a complex compound of gadolinium and manganese such as Gd-DTPA, Gd-DTPA-BMA, GdDOTA and Gd-DO3A, iron oxide as a superparamagnetic material, and one or more radioisotope selected from ¹⁸F, ¹²⁴I, ⁶⁴Cu, ^{99m}Tc and¹¹1n as a PET contrast agent, and may be encapsulated in the core or shell of the peptidesome in the form of a DOTA or DTPA complex depending on its hydrophilic or hydrophobic property. Specifically, a hydrophilic contrast agent may be encapsulated in the core of the peptidesome according to the present disclosure, and a hydrophobic contrast agent may be encapsulated in the shell having a bilayer structure. Since it is encapsulated spontaneously through self-assembly, the preparation process is easy and convenient and the synthesis yield is high. In addition, since the contrast agent can be delivered effectively into the target cell without additional carrier, cancer can be diagnosed and detected accurately.

The composition for diagnosing cancer according to the present disclosure can be administered to a living organism or a sample and an image may be obtained by detecting fluorescence signals from the living organism or the sample. The fluorescence from the living organism or sample may be analyzed to provide information for diagnosing cancer including the location of cancer cells.

In the present disclosure, the “sample” refers to a tissue or a cell isolated from a subject of diagnosis. The step of administering the composition for diagnosing cancer to a living organism or a sample may be carried out through a route commonly used in the medical field. For example, an oral administration route or a parenteral administration such as intravenous, intraperitoneal, intramuscular, subcutaneous or topical routes may be used.

Hereinafter, the present disclosure will be described in more detail through specific examples. However, the examples are only for describing the present disclosure more specifically and it will be obvious to those having ordinary knowledge in the art that the scope of the present disclosure is not limited by them.

<Experimental Materials>

General Chemicals were purchased from Sigma-Aldrich (USA) and Merck (Germany). Fmoc-amino acids and coupling reagents were purchased from Novabiochem (Germany) and Anaspec (USA). Fmoc-PEG2-Suc-OH (Cat number: AS-61924-1) was purchased from Anaspec. Fmoc-PEG2-Suc-OH is also called Fmoc-Ebes-OH (oligoethylene glycol-based linker N-(Fmoc-8-amino-3,6-dioxaoctyl)succinamic acid). HPLC solvents and culture media were purchased from Fisher Scientific (USA), pheophorbide from Cayman (USA), MTT (thiazoyl blue tetrazolium bromide) from Biosesang (Seongnam, Gyeonggi-do, Korea) and Hoechst 33342 from Thermo Fisher Scientific (Waltham, MA, USA).

The size of self-assembled nanoparticles (SNPs) was analyzed by a dynamic light scattering size distributor (particle size & zeta potential analyzer, ELS-1000ZS, Otsuka Electronics, Japan) using a UV-transparent cuvette having a path length of 1 cm. The secondary structure of the cyclic peptides of SPNs was analyzed using a Chirascan circular dichroism spectrometer equipped with a Peltier temperature controller (Applied Photophysics, UK). The CD (circular dichroism) spectra of samples were analyzed in a range of 190-260 nm using a cuvette having a path length of 2 mm. The molar residue ellipticity of the samples was calculated per amino acid residue. All mouse experiments were conducted under an animal protocol approved by the Catholic University of Korea on Laboratory Animal Care (2020-0359-05).

<Experimental Methods>

Atomic force microscopy (AFM)

5 μL of a sample was placed onto a freshly cleaved mica surface and dried. When a salt was present in the sample, the excess salt was removed by washing with 3 mL of distilled water (DW). Then, the excess distilled water was wicked off and the sample was dried quickly under argon atmosphere. The dried sample was analyzed using an NX10 AFM instrument (Park Systems, Korea) in noncontact mode. Scan rate was set to 1.0 Hz. The data were analyzed using the XEN software.

Transmission Electron Microscopy (TEM)

3 μL of a sample was placed on a carbon-coated copper grid. After 1 minute, the excess sample was wicked off with a filter paper. For negative staining, a 1-2 mL drop of 0.1% (w/v) uranyl acetate/distilled water was added to the grid. After 1 minute, the excess staining solution was wicked off with a filter paper. The stained sample was analyzed using a JEM-F200 field emission transmission electron microscope (JEOL, Japan) at an accelerating voltage of 200 kV. The data were analyzed using the GATAN software.

Analysis of intracellular delivery efficiency and FACS in vitro Cellular uptake of materials in SCC7 cells was analyzed based on the intrinsic fluorescence of Pa via CLSM (LSM700, Carl Zeiss, Germany) and flow cytometry (FACS Canto II, BD Biosciences, Bedford, MA, USA). SCC7 cells were seeded onto a 24-well plate at a density of 5×10⁴ and incubated overnight. Then, 200 mL of a sample was added to 800 mL of a culture medium for each well. The cells were treated with the sample for 4 hours. Then, the sample was removed and the cells were washed. For cell imaging, cell nuclei were stained with Hoechst 33342.

Detection of ROS generation ROS production in vivo and in vitro was measured using DCFDA (2′,7′-dichlorofluorescin diacetate). SCC7 cells were treated with a sample at 2 μg/mL for 1 hour and washed out. The washed cells were treated with 20 μM DCFDA dissolved in PBS for 30 minutes and then laser was irradiated to the cells. DCFDA fluorescence of the cells was measured in the FITC wavelength by flow cytometry. For in-vivo ROS measurement, 50 mg/kg of DCFDA was intratumorally injected into tumor-bearing mice and then PDT was performed as previously reported [S. Uthaman, S. Pillarisetti, A. P. Mathew, Y Kim, W K. Bae, K. M. Huh, I. K. Park, Long circulating photoactivable nanomicelles with tumor localized activation and ROS triggered self-accelerating drug release for enhanced locoregional chemo-photodynamic therapy, Biomaterials 232 (2020)]. Tumor tissue was excised from the mice and cryosections were prepared with a thickness of 10 μm, followed by detecting the fluorescence of DCFDA by inverted fluorescence microscopy.

Analysis of in-vivo biodistribution Tumor-bearing mice were developed by subcutaneously injecting 2×10⁶ SCC7 tumor cells in 30 mL of saline into the left thigh of C3H/HeN mice. When the tumor size reached 200-250 mm³, 100 mL of the sample solution was administered via the tail vein. Whole-body biodistribution was observed at 3 hours, 6 hours, 12 hours and 24 hours after the injection of the sample using IVIS Lumina XRMS (PerkinElmer, Inc., Waltham, MA, USA). At each time point, 30 μL of blood was collected from the tail vein and then fluorescence imaging was performed using an IVIS system. At 24 hours, tumors and major organs (heart, lung, liver, spleen and kidney) were dissected and ex-vivo fluorescence images were obtained by IVIS. IVIS imaging was performed at the wavelength of Cy5.5. The dissected tumors were fixed in 4% paraformaldehyde for 24 hours and treated with increasing concentrations of sucrose from 10% to 20%. Then, the tumor tissues were frozen in OCT (optimal cutting temperature) compound and sectioned to 10 μm thickness. The sectioned tissues were attached to a glass slide and dried. The dried tissues were washed several times with PBS and stained with 2 μg/mL of Hoechst 33342 at room temperature for 20 minutes. The fluorescence from the tissues was observed with an Observer.Z1 inverted fluorescence microscope (Carl Zeiss, Jena, Germany).

Analysis of In-Vivo Antitumor Efficacy

SCC7 tumor-bearing mouse models were prepared similarly to the biodistribution analysis. When the tumor volume reached approximately 50 mm³, the sample was intravenously injected into the xenograft mice. 3 hours after the sample injection, NIR laser (671 nm) was irradiated to the tumor site with a power of 0.53 W/cm² for 15 minutes, and the same sample injection and NIR irradiation were repeated on the next day. The tumor volume and body weight were recorded every two days. At the end of the therapy, major organs and tumors were dissected for histological analysis. The organs and tumor tissues were fixed, sliced, stained with hematoxylin and eosin (H&E), and observed with a microscope (Axiolmager A1, Zeiss, Germany).

Statistical Analysis

Student's t-test was used to compare the differences between two groups. One-way analysis of variance (ANOVA) and Tukey's post hoc analysis were used to compare differences among multiple groups. P<0.05 was considered statistically significant.

### Examples 1-1 to 1-4. Synthesis of Linear Peptides

The first residue (Fmoc-Ebes-OH) was loaded on 2-chlorotrityl resin (Novabiochem, Germany) in 1 M DIPEA/MC (diisopropylethylamid/methylene chloride). Then, amino acid coupling was performed using the standard Fmoc protocol in a Tribute peptide synthesizer (Protein Technologies, USA). Standard amino acid protecting groups were used for the synthesis except for Dde-Lys(Fmoc)-OH. To prepare a protected fragment, the N-terminal Fmoc group was removed. Then, the peptide-loaded resin was treated with a cleavage cocktail (acetic acid/TFE (2,2,2-trifluoroethanol)/MC (2:2:6, v/v/v)) for 1-2 hours, filtered and collected (4 mL×2 cycles). Acetic acid was removed as an azeotrope with hexane to obtain linear peptides represented by Structural Formulas 1-4 in the form of white powder.

[Structural Formula 1]
W-ϵ-KW-linker-RR-linker
[Structural Formula 2]
W-ϵ-KW-linker-RRR-linker
[Structural Formula 3]
W-ϵ-KW-linker-RRRRRR-linker
[Structural Formula 4]
W-ϵ-KW-linker-RGDRGD-linker

In the above formulas, the linker is a compound represented by Structural Formula a (Ebes), and ε indicates the epsilon-amino group of the lysine residue.

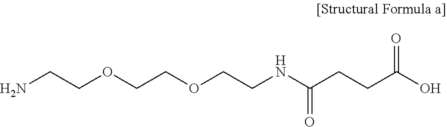 [SMILES: NCCOCCOCCNC(=O)CCC(=O)O]

### Example 2-1. Synthesis of Cyclic Peptide (R₂)

A cyclic peptide was prepared using the linear peptide of Example 1-1. First, a pseudo-high-dilution condition for head-to-tail cyclization was achieved using a dual syringe method. One syringe was filled with one of the linear peptides of Examples 1-1 to 1-4 (20 μmol, 1 eq) and DIPEA (4 eq) in DMF (20 mL) while the other syringe was filled with HCTU (2-(6-chloro-1H-benzotriazole-1-yl)-1,1,3,3-tetramethylaminium hexafluorophosphate, 1 eq) dissolved in DMF (20 mL). The solutions in the two syringes were added to a round-bottomed flask containing HCTU (0.1 eq) and HOBt (hydroxybenzotriazole, 1 eq) in DMF (20 mL) at a rate of 0.06 mL/min using a syringe pump. After the completion of the syringe injection, the reaction mixture was stirred overnight at 55° C. Then, DMF was removed by rotary evaporation and the cyclized peptide was precipitated by adding a mixture of MC, TBME (tert-butyl methyl ether) and hexane. The Dde group in lysine was deprotected using 2% (v/v) hydrazine/DMF (2 min×4 cycles).

To conjugate a fatty acid (lauric acid) tail to the prepared cyclic peptide, the cyclized peptide fragment (20 μmol, 1 eq), lauric acid (5 eq) and DIPEA (10 eq) were dissolved in DMF (2 mL) and stirred overnight. The product was precipitated with distilled water (DW) and recovered through centrifugation. The final deprotection was performed in a cleavage cocktail (TFA/TIS/water; 95:2.5:2.5, v/v/v) for 3 hours, followed by trituration with TBME, to prepare a fatty acid-conjugated cyclic peptide represented by Chemical Formula 1 (R₂).

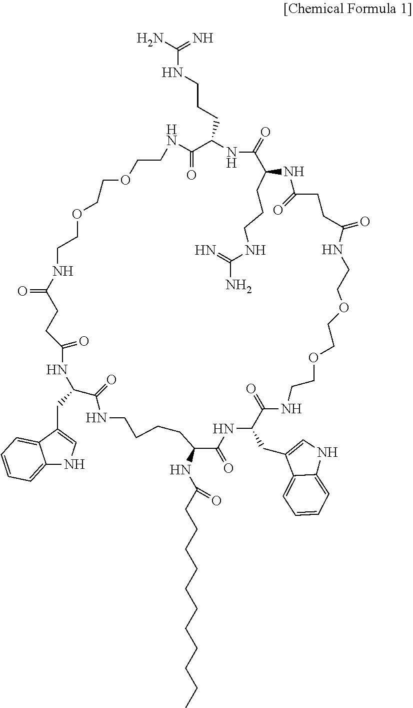 [SMILES: CCCCCCCCCCCC(=O)N[C@H]1CCCCNC(=O)[C@H](Cc2c[nH]c3ccccc23)NC(=O)CCC(=O)NCCOCCOCCNC(=O)[C@H](CCCNC(=N)N)NC(=O)[C@H](CCCNC(=N)N)NC(=O)CCC(=O)NCCOCCOCCNC(=O)[C@H](Cc2c[nH]c3ccccc23)NC1=O]

The prepared cyclic peptide was purified by reversed-phase high-performance liquid chromatography (HPLC) using water (0.1% TFA) and acetonitrile (0.1% TFA) as eluents.

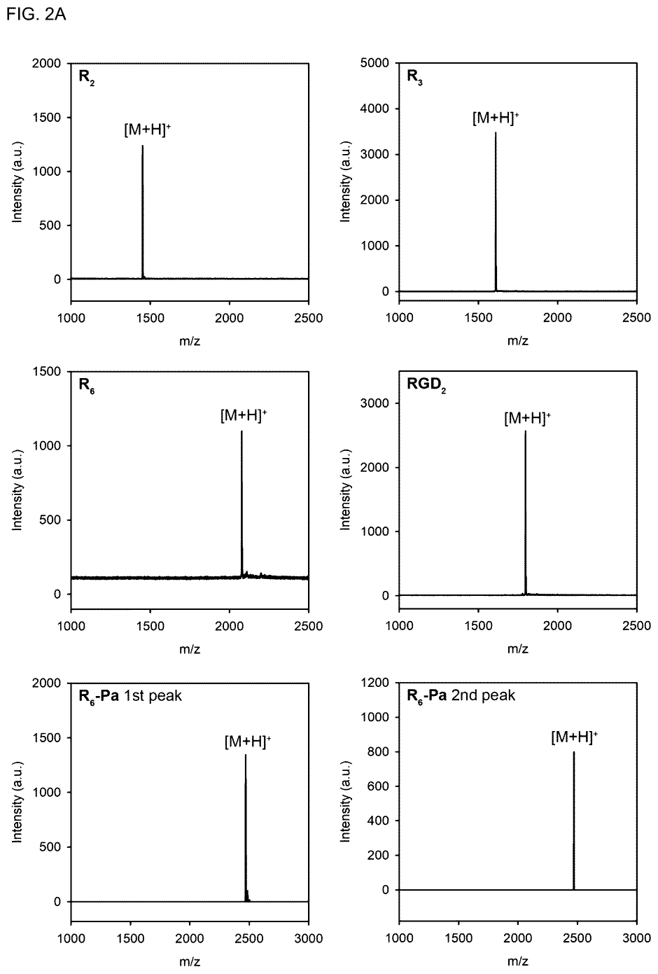

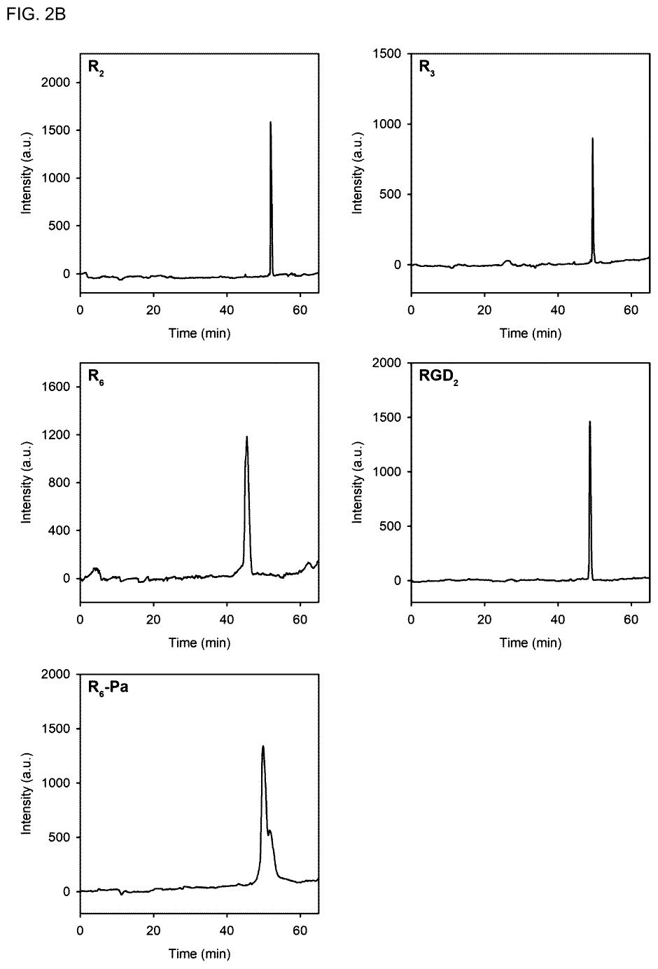

The molecular weight of the prepared cyclic peptide was investigated by MALDI-TOF (matrix-assisted laser desorption/ionization time-of-flight) mass spectrometry and the result is shown in FIG. 2a. The purity of the peptide was >95% as determined by analytical HPLC. The HPLC analysis result is shown in FIG. 2b. The concentration of the cyclic peptide was determined spectrophotometrically in water/acetonitrile (1:1) using the molar extinction coefficient of tryptophan (5,502 M⁻¹cm⁻¹) at 667 nm and Pa (44,500 M⁻¹cm⁻¹) at 280 nm for Rn and R₆-Pa, respectively.

### Example 2-2. Synthesis of Cyclic Peptide (R₃)

A fatty acid-conjugated cyclic peptide represented by Chemical Formula 2 (R₃) was prepared in the same manner as in Example 2-1 except that the linear peptide of Example 1-2, rather than Example 1-1, was used.

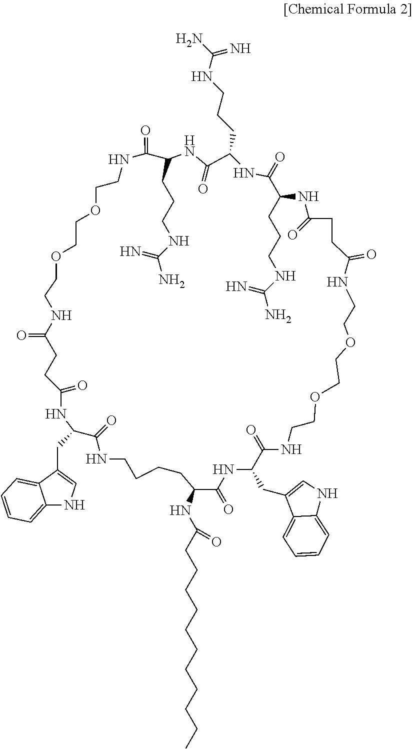

The prepared cyclic peptide was purified by reversed-phase high-performance liquid chromatography (HPLC) using water (0.1% TFA) and acetonitrile (0.1% TFA) as eluents.

The molecular weight of the prepared cyclic peptide was investigated by MALDI-TOF (matrix-assisted laser desorption/ionization time-of-flight) mass spectrometry and the result is shown in FIG. 2a. The purity of the peptide was >95% as determined by analytical HPLC. The HPLC analysis result is shown in FIG. 2b. The concentration of the cyclic peptide was determined spectrophotometrically in water/acetonitrile (1:1) using the molar extinction coefficient of tryptophan (5,502 M⁻¹cm⁻¹) at 667 nm and Pa (44,500 M⁻¹cm⁻¹) at 280 nm for Rn or R₆-Pa, respectively.

### Example 2-3. Synthesis of Cyclic Peptide (R₆)

A fatty acid-conjugated cyclic peptide represented by Chemical Formula 3 (R₆) was prepared in the same manner as in Example 2-1 except that the linear peptide of Example 1-3, rather than Example 1-1, was used.

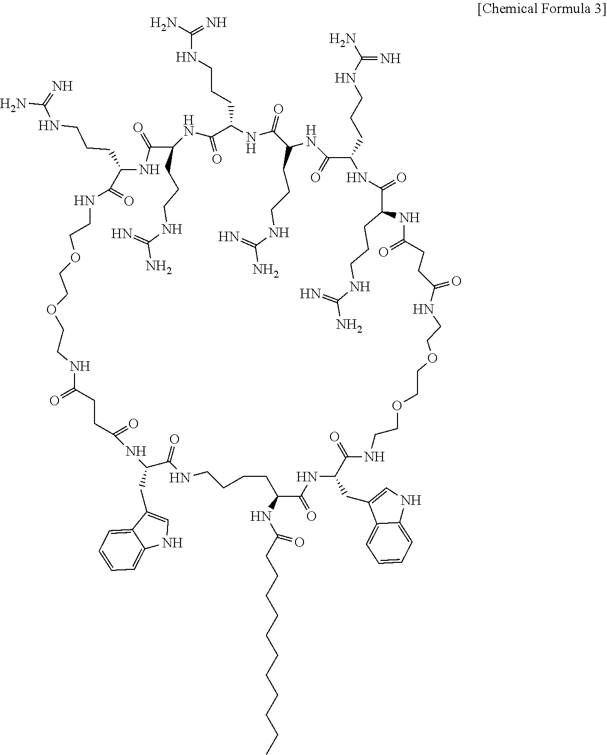

The prepared cyclic peptide was purified by reversed-phase high-performance liquid chromatography (HPLC) using water (0.1% TFA) and acetonitrile (0.1% TFA) as eluents.

The molecular weight of the prepared cyclic peptide was investigated by MALDI-TOF (matrix-assisted laser desorption/ionization time-of-flight) mass spectrometry and the result is shown in FIG. 2a. The purity of the peptide was >95% as determined by analytical HPLC. The HPLC analysis result is shown in FIG. 2b. The concentration of the cyclic peptide was determined spectrophotometrically in water/acetonitrile (1:1) using the molar extinction coefficient of tryptophan (5,502 M⁻¹cm⁻¹) at 667 nm and Pa (44,500 M⁻¹cm⁻¹) at 280 nm for Rn or R₆-Pa, respectively.

### Example 2-4. Synthesis of Cyclic Peptide (RGD₂)

A fatty acid-conjugated cyclic peptide represented by Chemical Formula 4 (RGD₂) was prepared in the same manner as in Example 2-1 except that the linear peptide of Example 1-4, rather than Example 1-1, was used.

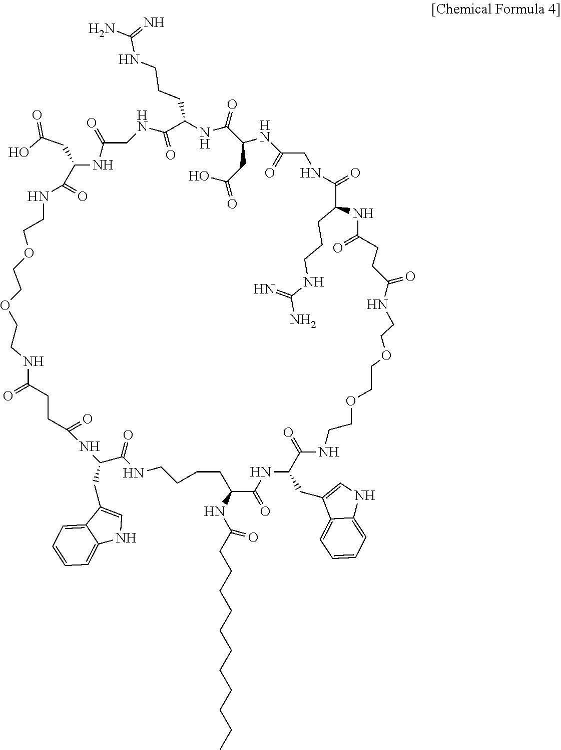 [SMILES: CCCCCCCCCCCC(=O)N[C@H]1CCCCNC(=O)[C@H](Cc2c[nH]c3ccccc23)NC(=O)CCC(=O)NCCOCCOCCNC(=O)[C@H](CC(=O)O)NC(=O)CNC(=O)[C@H](CCCNC(=N)N)NC(=O)[C@H](CC(=O)O)NC(=O)CNC(=O)[C@H](CCCNC(=N)N)NC(=O)CCC(=O)NCCOCCOCCNC(=O)[C@H](Cc2c[nH]c3ccccc23)NC1=O]

The prepared cyclic peptide was purified by reversed-phase high-performance liquid chromatography (HPLC) using water (0.1% TFA) and acetonitrile (0.1% TFA) as eluents.

The molecular weight of the prepared cyclic peptide was investigated by MALDI-TOF (matrix-assisted laser desorption/ionization time-of-flight) mass spectrometry and the result is shown in FIG. 2a. The purity of the peptide was >95% as determined by analytical HPLC. The HPLC analysis result is shown in FIG. 2b. The concentration of the cyclic peptide was determined spectrophotometrically in water/acetonitrile (1:1) using the molar extinction coefficient of tryptophan (5,502 M⁻¹cm⁻¹) at 667 nm and Pa (44,500 M⁻¹cm⁻¹) at 280 nm for Rn or R₆-Pa, respectively.

### Example 2-5. Synthesis of Cyclic Peptide (R₆-Pa)

The same procedure of Example 2-1 was conducted except that the linear peptide of Example 1-3, rather than Example 1-1, was used and the photodynamic therapy (PDT) agent Pa was conjugated instead of lauric acid.

After preparing a cyclic peptide from the linear peptide of Example 1-3 (cf. Example 2-1), the following procedure was conducted to conjugate Pa (pheophorbide a) instead of the fatty acid. A succinimidyl ester (NHS ester) of Pa was prepared first. The NHS ester of Pa was prepared by dissolving Pa (20 mg, 1 eq), NHS (N-hydroxysuccinimide) (1.7 eq), EDC (1-ethyl-3-(3-dimethylaminopropyl)carbodiimide hydrochloride) (1.7 eq) and DMAP (4-dimethylaminopyridine) (0.4 eq) in MC (6 mL) and conducting reaction overnight in the dark. The cyclized peptide fragment (25 μmol, 1 eq), the NHS ester of Pa (7 eq), triethylamine (14 eq), EDC (14 eq) and DMAP (14 eq) were dissolved in MC (5 mL) and reacted for 2 days. After evaporating MC from the reaction mixture, the obtained powder was redissolved in a small volume of MC and then precipitated using a mixture of TBME and hexane. The final deprotection was performed in a cleavage cocktail (TFA/TIS/water; 95:2.5:2.5, v/v/v) for 3 hours, followed by trituration with TBME, to prepare a fatty acid-conjugated cyclic peptide represented by Chemical Formula 5 (R₆-Pa).

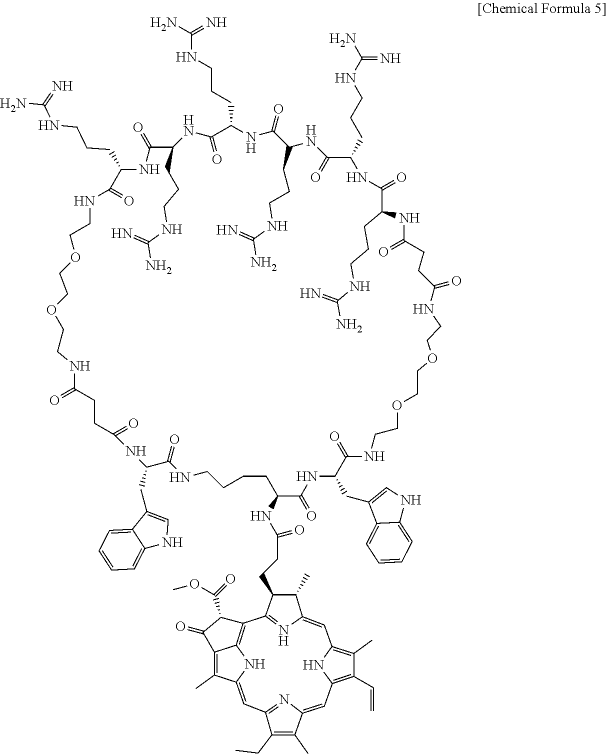

The prepared cyclic peptide was purified by reversed-phase high-performance liquid chromatography (HPLC) using water (0.1% TFA) and acetonitrile (0.1% TFA) as eluents.

The molecular weight of the prepared cyclic peptide was investigated by MALDI-TOF (matrix-assisted laser desorption/ionization time-of-flight) mass spectrometry and the result is shown in FIG. 2a. The purity of the peptide was >95% as determined by analytical HPLC. The HPLC analysis result is shown in FIG. 2b. Although two peaks were detected for R₆-Pa of Example 2-5, it is thought that they are attributable to the same compound in different forms. The concentration of the cyclic peptide was determined spectrophotometrically in water/acetonitrile (1:1) using the molar extinction coefficient of tryptophan (5,502 M^{31 1}cm⁻¹) at 667 nm and Pa (44,500 M^{31 1}cm⁻¹) at 280 nm for Rn or R₆-Pa, respectively.

### Examples 3-1 to 3-5. Preparation of Peptidesomes (R₂, R₃, R₆, RGD₂ and R₆-Pa)

The cyclic peptides prepared in Examples 2-1 to 2-5 were dissolved in a 30% (v/v) HFIP (hexafluoroisopropanol) aqueous solution to promote disassembly and molecular mixing. After evaporating the solvent from the mixture solution, the cyclic peptide was rehydrated using an appropriate solvent or buffer to induce self-assembly. Through this, vesicular peptidesomes (Examples 3-1 to 35) (R₂, R₃, R₆, RGD₂ and R₆-Pa) were prepared.

### Example 3-6. Preparation of Coassembled Peptidesome (R₆:RGD₂) (1:1)

The cyclic peptides prepared in Example 2-3 and Example 2-4 were dissolved in a 30% (v/v) HFIP aqueous solution at 50 mol %: 50 mol %. After evaporating the solvent from the mixture solution, the cyclic peptide was rehydrated using an appropriate solvent or buffer to induce co-self-coassembly. Through this, a vesicular peptidesome (R₆:RGD₂) (1:1) was prepared.

### Examples 4-1 to 4-4. Preparation of Drug-Encapsulated Peptidesomes (R₂<-Pa, R₃<-Pa, R₆<-Pa and RGD₂<-Pa)

Drug-encapsulated vesicular peptidesomes (R₂<-Pa of Example 4-1; R₃<-Pa of Example 4-2; R₆<-Pa of Example 4-3, RGD₂<-Pa of Example 4-4) were prepared by dissolving the cyclic peptides prepared in Examples 2-1 to 2-4 and Pa in a 30% (v/v) HFIP aqueous solution, followed by evaporation of the solvent and rehydration.

### Example 4-6. Preparation of Drug-Encapsulated Coassembled Peptidesome (R₆:RGD₂<-Pa)

The cyclic peptides prepared in Example 2-3 and Example 2-4 were dissolved in a 30% (v/v) HFIP aqueous solution at different ratios (Table 1). After evaporating the solvent from the mixture solution, the cyclic peptide was rehydrated using an appropriate solvent or buffer to induce co-self-coassembly. Unless specified otherwise, the solvent is distilled water (DW). Through this, a vesicular peptidesome (R₆:RGD₂<-Pa) (1:1-1:9) was prepared.

TABLE 1

| | Mixing ratio (mol %) | | Molar parts of Pa based on 100 |
|---|---|---|---|
| | Cyclic peptide | Cyclic peptide | molar parts of peptidesome |
| | of Example 2-3 | of Example 2-4 | Pa |
| Example 4-6a | 5 | 95 | 12 |
| Example 4-6b | 10 | 90 | 3.125 |
| Example 4-6c | | | 6.25 |
| Example 4-6d | | | 12 |
| Example 4-6e | | | 25 |
| Example 4-6f | | | 50 |
| Example 4-6g | | | 100 |
| Example 4-6h | | | 200 |
| Example 4-6i | 15 | 85 | 12 |
| Example 4-6j | 50 | 50 | 12 |

### Test Example 1. Structural Analysis of Cyclic Peptides

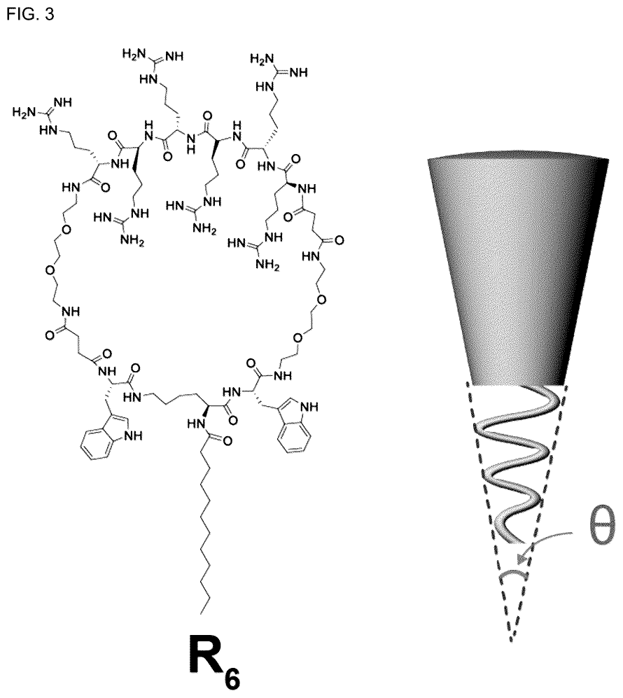

The cyclic peptide according to the present disclosure has a hydrophilic segment consisting of arginine residues and a hydrophobic segment consisting of two tryptophan residues and a C₁₂ hydrocarbon compound. The structure of the cyclic peptide (R₆) is schematically shown in FIG. 3.

Initially, the self-assembly behavior of the cyclic peptide having two arginine residues prepared in Example 2-1 was investigated. Then, the self-assembly behavior of the cyclic peptides of Examples 2-1 to 2-3 was investigated. Specifically, each cyclic peptide (R₂, R₃, R₆) was dissolved in distilled water (DWV) and then analyzed by probe sonication.

FIG. 4 is an AFM image showing the self-assembly behavior of the cyclic peptide of Example 2-1 (R₂) in distilled water. The structure of a peptidesome prepared as the cyclic peptide (R₂) self-assembles into a vesicle in a liquid is shown at the top of FIG. 4. From FIG. 4, it can be seen that the cyclic peptide (R₂) self-assembles into a spherical vesicle in distilled water.

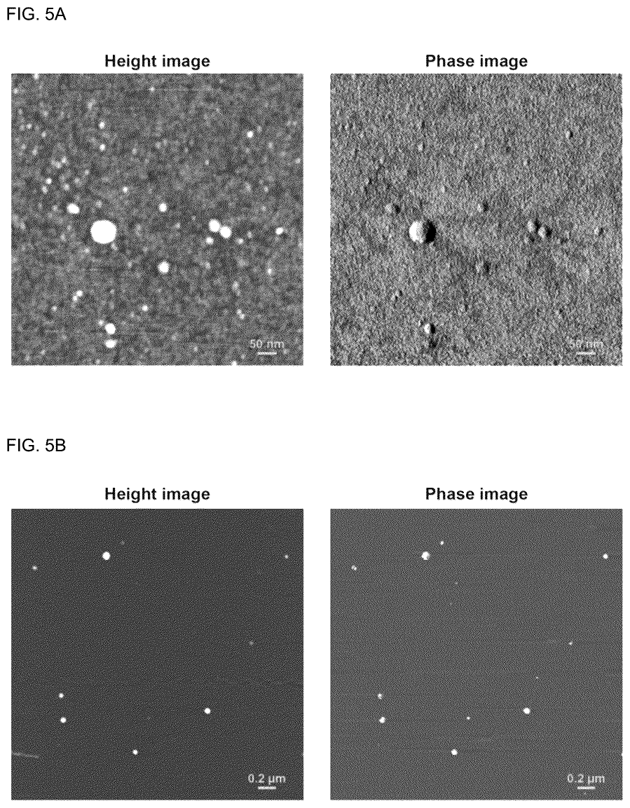

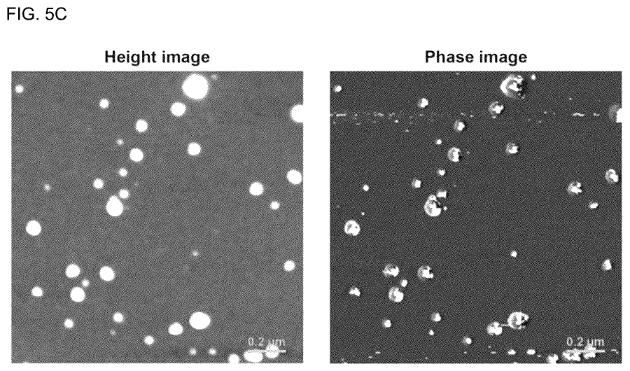

FIG. 5A shows AFM images showing the self-assembly behavior of the cyclic peptide of Example 2-1 (R₂), FIG. 5B shows the cyclic peptide of Example 2-2 (R₃) and FIG. 5C shows the cyclic peptide of Example 2-3 (R₆) in distilled water.

More arginine residues were added to increase the volume fraction of the hydrophilic segments in the cyclic peptides of Example 2-2 and Example 2-3 while maintaining the basic structure of the cyclic peptide of Example 2-1. As a result of measuring the morphology of the peptidesomes prepared therefrom (R₃, R₆) by AFM, it can be seen that all the cyclic peptides according to the present disclosure self-assembled into peptidesomes (vesicles) regardless of the length of the arginine residues.

### Test Example 2. Average Diameter of Peptidesomes

It was confirmed from the foregoing experiment that the cyclic peptides prepared in Examples 2-1 to 2-3 form vesicular self-assembled structure when stored in distilled water (DW). The average hydrodynamic diameter (Dn) of the self-assembled structures was measured by dynamic light scattering (DLS) at different temperatures (20° C., 30° C., 40° C.).

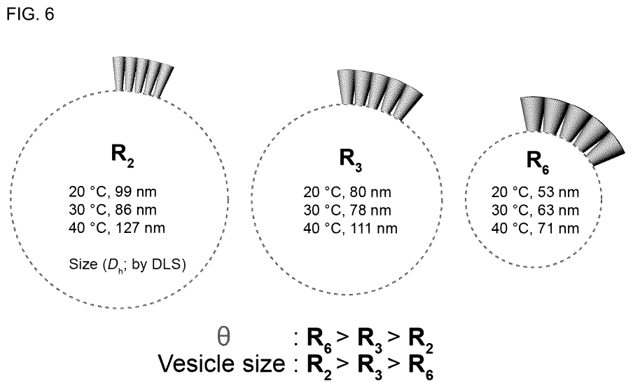

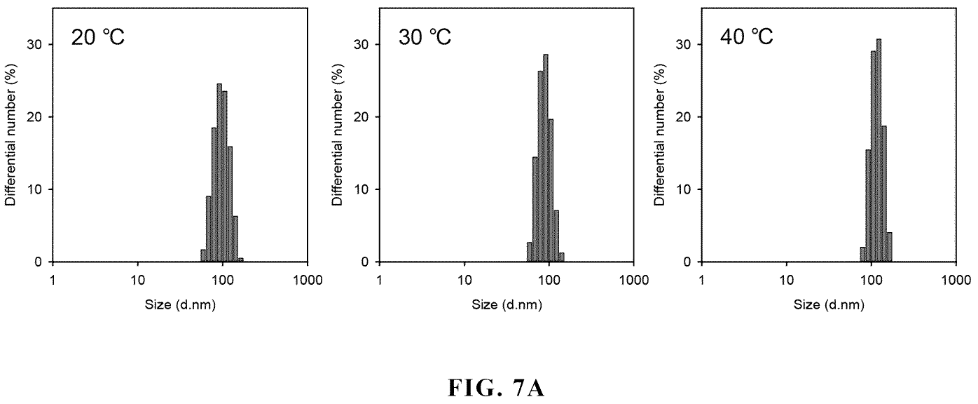

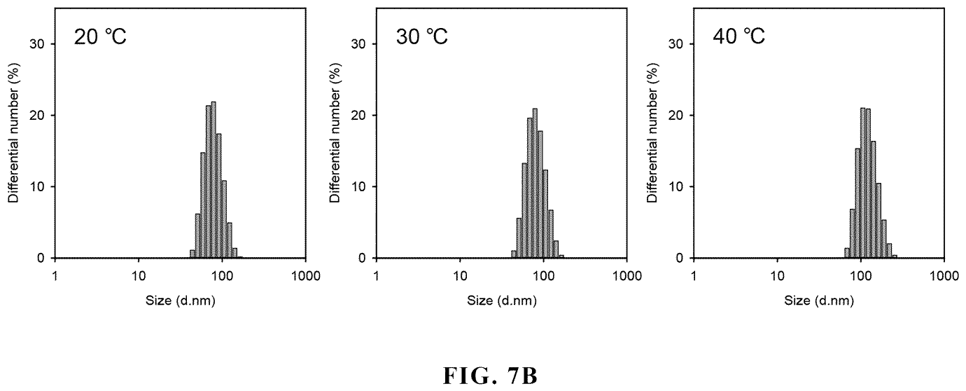

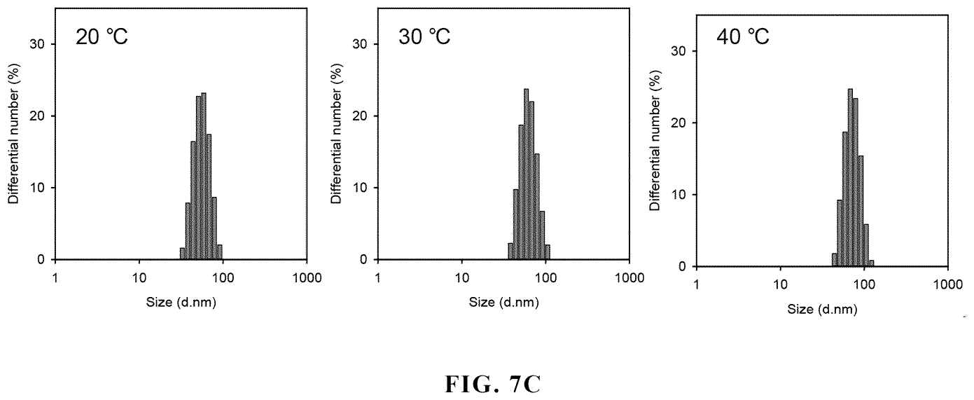

FIG. 6 shows the structure and average diameter of the peptidesomes, which are self-assembled nanostructures, in distilled water depending on the cone angle of the cyclic peptides prepared in Examples 2-1 to 2-3 and temperature. FIGS. 7A to 7C show results of measuring the average diameter of the peptidesomes prepared from the cyclic peptides of Example 2-1 (FIG. 7A), Example 2-2 (FIG. 7B) and Example 2-3 (FIG. 7C) depending on temperature (20° C., 30° C. and 40° C.) by DLS.

The self-assembled morphology of the cyclic peptide of the present disclosure is influenced by the packing parameter (P) and molecular shape. Therefore, in the present disclosure, the shape of the cyclic peptide as a building block to form the peptidesome was simplified to a cone. Accordingly, it was anticipated that the cyclic peptide according to the present disclosure would self-assemble to form a vesicular nanostructure having a shell with a bilayer structure in a liquid, and it was named peptidesome.

It was expected that the cone angle of the cyclic peptide according to the present disclosure would have an influence on the final size of the peptidesome. In order to confirm this, the average diameter was measured at different temperatures by DLS and the result is schematically shown in FIG. 6.

As shown in FIG. 6, it was confirmed that the cyclic peptide according to the present disclosure behaves as a building block in a fluid and forms a peptidesome, which is a vesicular, spherical self-assembled nanostructure. It was confirmed that the peptidesomes prepared from the cyclic peptides having different cone angles have different average diameters.

From FIG. 6 and FIGS. 7A to 7C, it can be seen that the peptidesome formed from the cyclic peptide of Example 2-1 has an average diameter of 99-127 nm in distilled water at 20-40° C. In general, spherical micelles are homogeneous in size and their diameters are twice as large as the molecular length. Considering that the molecular length of the cyclic peptide according to the present disclosure (R₂) is 4.5 nm, it can be seen that the peptidesome (R₂) prepared therefrom is in the form of a vesicle, not a micelle. In addition, it was confirmed that peptidesomes with a size of 100 nm or smaller can be prepared easily through self-assembly of the cyclic peptides of Examples 2-1 to 2-3 in distilled water.

To conclude, it can be seen that the cyclic peptide according to the present disclosure self-assembles to form a peptidesome having a vesicular structure, and the size of the peptidesome decreases as the volume fraction of arginine residues in the building block increases. In particular, the size of the peptidesome (R₆) prepared from the cyclic peptide having six arginine residues was sufficiently smaller than 100 nm at all temperatures.

### Test Example 3. Structural Analysis of Peptidesome

The cyclic peptide prepared in Example 2-3 was added to distilled water (DW) and a peptidesome prepared therefrom (R₆) was imaged by TEM for structural analysis.

FIGS. 8A and 8B shows the TEM images of the peptidesome (R₆) prepared from the cyclic peptide of Example 2-3. It can be seen that the peptidesome (R₆) has a spherical, vesicular structure having a shell layer with a bilayer structure and a hollow core. The shell layer had a thickness of 10.26±0.68 nm, which matched well with the bilayer model predicted from the cyclic peptide (R₆) (˜9.92 nm).

### Test Example 4. Structural Analysis of Peptidesome Depending on Drug Loading

According to the Lipinski's rule of five for evaluating the similarity of drugs, most drugs are lipophilic. Pa (pheophorbide a), which is a lipophilic drug, was used to evaluate the efficacy of the peptidesome of the present disclosure as a drug carrier. Pa is also known as a hydrophobic material with a low water solubility of 0.014 g/L. Pa is a porphyrin derivative of plant chlorophyll and has been used as a photosensitizer for PDT (photodynamic therapy).

For noncovalent drug loading in the peptidesome (R₆), the cyclic peptide of Example 2-3 and Pa were mixed in distilled water and the solution was sonicated vigorously for 1 minute to prepare a drug-encapsulated peptidesome (Peptidesome R₆<-Pa). The peptidesome was imaged by AFM before and after the sonication for comparison.

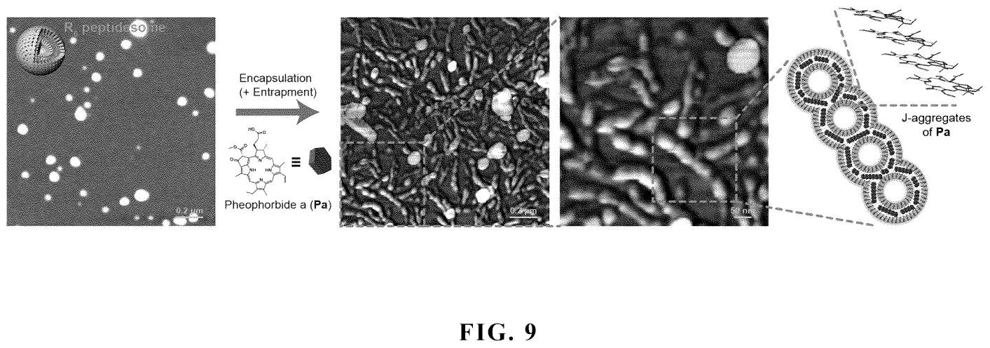

FIG. 9 shows the AFM images and structure of the peptidesome before encapsulation of the drug (R₆) and after encapsulation of the drug (R₆<-Pa). It can be seen that the peptidesome (R₆) which maintained a spherical shape in the solution underwent morphological transformation to an elongated superstructure after the loading of Pa.

After the drug loading, the peptidesome (R₆<-Pa) has a peapod-like elongated superstructure with an uneven surface through fusion with nearby peptidesomes (R₆). The thickness of the elongated superstructure coincided with those of adjacent vesicles.

It was confirmed that the hydrophobic drug Pa was mostly encapsulated in the shell layer having a bilayer structure, while a minor portion of solvated Pa molecules were entrapped in the hydrophobic core.

### Test Example 4. Stability of Drug-Encapsulated Peptidesome of Example 4-3 (R₆<-Pa)

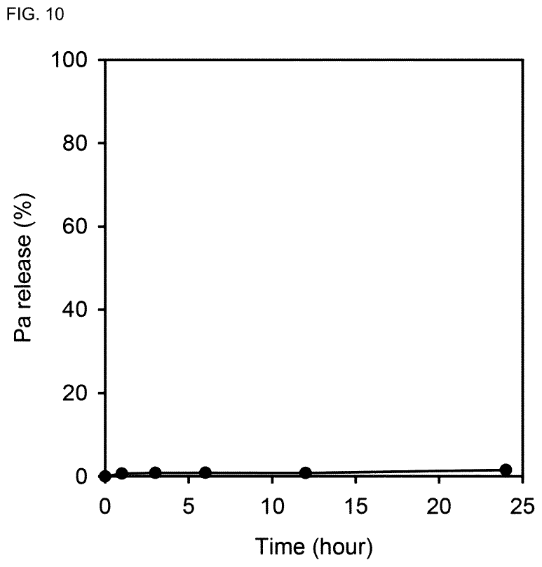

FIG. 10 shows a result of analyzing the release of Pa (%) at 37° C. with time when the drug-encapsulated peptidesome of Example 4-3 (R₆<-Pa) was stored in PBS containing 2% (w/v) Tween 80.

As shown in FIG. 10, the drug-encapsulated peptidesome of Example 4-3 (R₆<-Pa) was stable in vitro for 24 hours without drug release. Accordingly, it can be seen that the drug-encapsulated peptidesome according to the present disclosure can be stored for a long time at room temperature without drug release.

### Test Example 5. Analysis of UV Absorption Spectrum of Drug-Encapsulated Peptidesome (R₆<-Pa)

For noncovalent drug loading in the peptidesome (R₆), the drug-encapsulated coassembled peptidesome of Example 4-6d (R₆:RGD₂<-Pa) (1:9) and free Pa were prepared and imaged by AFM for comparison. The drug-encapsulated coassembled peptidesome of Example 4-6d (R₆:RGD₂<-Pa) (1:9) was dissolved in distilled water and free Pa was dissolved in DMSO.

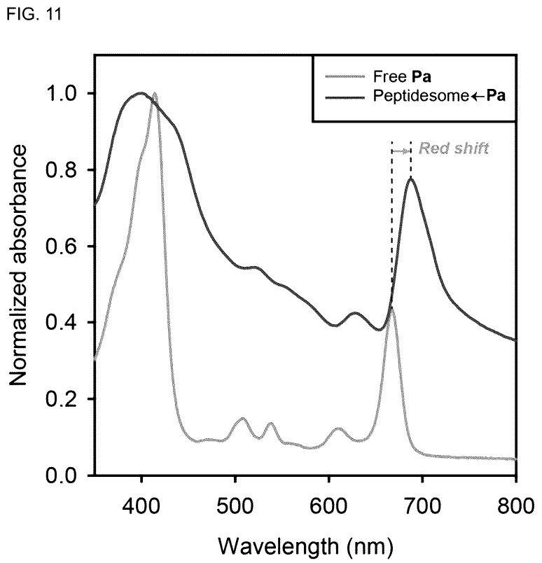

FIG. 11 shows the UV absorption spectra of free Pa and the drug-encapsulated coassembled peptidesome of Example 4-6d (R₆:RGD₂<-Pa) (1:9).

As shown in FIG. 11, redshift and increased absorption intensity were observed in the Q-band of free Pa. For the peptidesome of Example 4-6d (R₆:RGD₂<-Pa) (1:9), head-to-tail J-aggregation of Pa was observed within the shell having a bilayer structure. That is to say, it can be seen that interparticle fusion occurs in the peptidesome of Example 4-6d (R₆:RGD₂<-Pa) (1:9) as Pa molecules are connected through head-to-tail stacking.

### Test Example 6. Structural Analysis of Peptidesome Prepared from Cyclic Peptide of Example 2-5 (R₆-Pa)

To further corroborate the elongation mechanism of the peptidesome according to the present disclosure through interparticle fusion, analysis was conducted on a cyclic peptide wherein Pa is chemically conjugated to the hydrophobic segment of the cyclic peptide (Example 2-5; R₆-Pa). Specifically, after dispersing the cyclic peptide of Example 2-5 (R₆-Pa) in distilled water, the peptidesome formed through self-assembly was imaged by AFM.

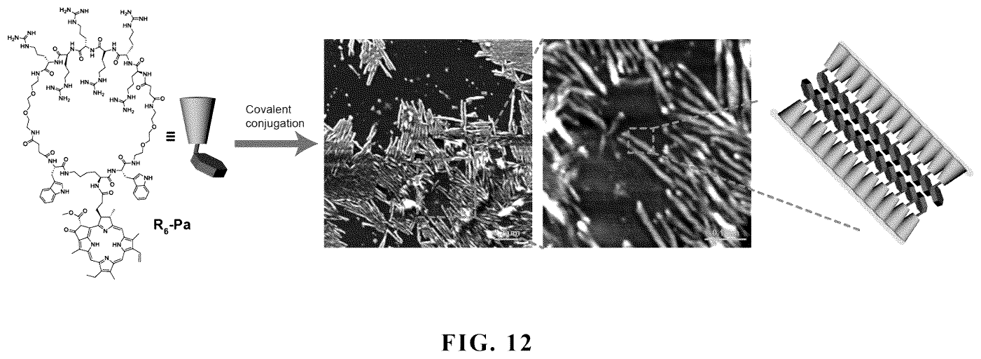

FIG. 12 shows the AFM image of the peptidesome prepared from the cyclic peptide of Example 2-5 (R₆-Pa). It was confirmed that the cyclic peptide of Example 2-5 (Re-Pa) has a shape similar to that of a peapod-like nanostructure with an uneven surface in distilled water, suggesting that the formation of a superstructure by the cyclic peptide according to the present disclosure can be controlled through drug loading.

### Test Example 7. In-Vivo Stability of Peptidesomes

A drug carrier should have high intracellular delivery efficiency and low toxicity such as side effects in vivo. In general, the two properties have inversely proportional relationship. For example, during cell entry of a drug carrier, certain parts of the cell need to be abnormally disrupted to achieve high intracellular delivery efficiency, which could result in side effects. Because in-vivo toxicity is the primary evaluation criterion in a phase 1 clinical trial, a toxic drug carrier cannot pass through this phase. Although the existing cell-penetrating peptides are designed based on several arginine residues, they exhibit the side effect of inducing the death of normal cells due to increased cytotoxicity.

Therefore, the cytotoxicity of the peptidesome according to the present disclosure (R₆) was investigated by analyzing the viability of cells treated therewith. To alleviate the toxicity caused by the arginine residues, a coassembled peptidesome was devised by mixing cyclic peptides having zwitterionic RGD as hydrophilic segments (Example 2-5, RGD₂) and cell viability was analyzed.

Specifically, SCC7 cells were treated with the peptidesome of Example 3-3 (R₆), the peptidesome of Example 3-4 (RGD₂) or the coassembled peptidesome of Example 3-6 (R₆:RGD₂) at different concentrations (1 μM, 3 μM, 6 μM, 9 μM, 15 μM, 30 μM) and incubated for 4 hours, and cell viability was measured by MTT assay.

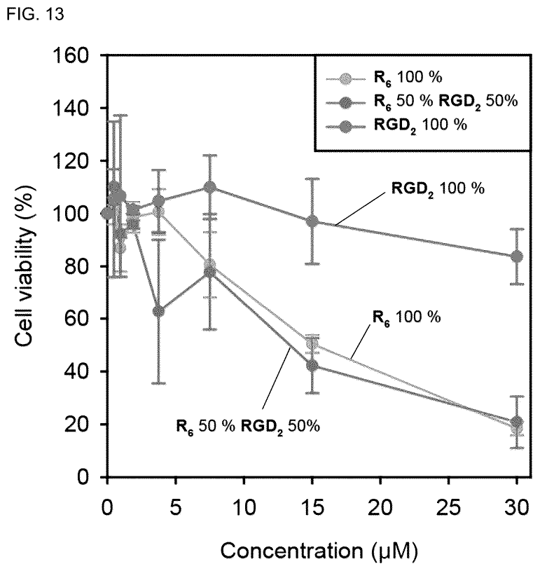

FIG. 13 shows a result of analyzing the cell viability for the peptidesome of Example 3-3 (R₆), the peptidesome of Example 3-4 (RGD₂) and the coassembled peptidesome of Example 3-6 (R₆:RGD₂) at different concentrations. It was confirmed that the peptidesome of Example 3-3 (R₆) and the coassembled peptidesome of Example 3-6 (R₆:RGD₂) exhibit cytotoxicity at 10 μM or higher. In contrast, the peptidesome of Example 3-4 (RGD₂) showed no cytotoxicity even at high concentrations.

### Test Example 8. In-Vivo Stability of Coassembled Peptidesomes

From the result of Test Example 7, it was confirmed that cytotoxicity can be controlled when preparing a coassembled peptidesome by mixing R₆ and RGD₂. Based on this result, coassembled peptidesomes were fabricated with varying proportions of R₆ and RGD₂, and then cytotoxicity and intracellular delivery efficiency were investigated. For preventing aggregate formation caused by Pa encapsulation, the proper drug loading range was determined by analyzing fluorescence spectra depending on the Pa loading concentration.

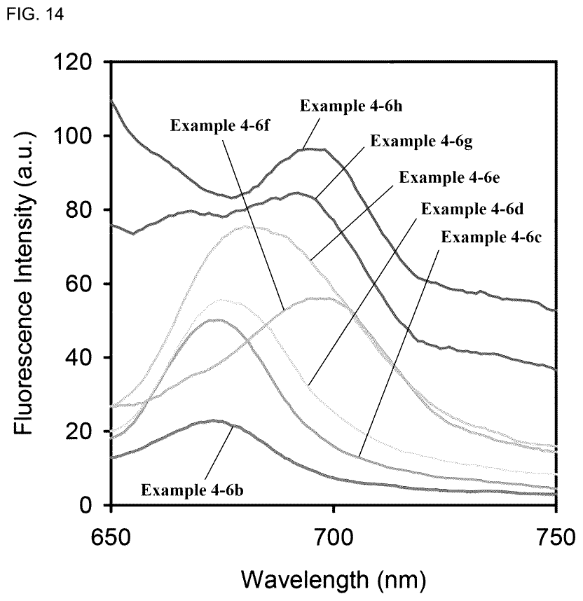

FIG. 14 shows the fluorescence spectra of the coassembled peptidesomes (R₆:RGD₂<-Pa) (Examples 4-6b to 4-6h) prepared varying the molar concentration of Pa. Excitation wavelength is 507 nm.

As shown in FIG. 14, the spectrum of the coassembled peptidesome (R₆:RGD₂<-Pa) (Example 4-6e) started to blueshift (674 nm→698 nm), and the spectrum of the coassembled peptidesome (R₆:RGD₂<-Pa) (Example 4-6f) was shifted completely. This verifies a J-aggregate was formed when the drug (Pa) was added at an amount of 50 molar parts or more based on 100 molar parts of the peptidesome.

Even though the Pa loading capacity could be increased to 200 molar parts or higher based on 100 molar parts of the peptidesome, the coassembled peptidesome (R₆:RGD₂<-Pa) (Example 4-6h) started to participate when the drug (Pa) loading was 200 molar parts based on 100 molar parts of the peptidesome. Therefore, it is preferred to load the drug (Pa) in an amount less than 25 molar parts based on 100 molar parts of the peptidesome in order to avoid vesicle elongation caused by J-aggregation.

Because Pa has strong hydrophobicity, it is encapsulated in the shell layer rather than in the core of the peptidesome. Accordingly, the loading efficacy of the peptidesome according to the present disclosure for a hydrophobic drug is nearly 100%.

### Test Example 9. Cell Viability Analysis for Coassembled Peptidesome (R₆:RGD₂<-Pa)

After adding the coassembled peptidesome of Example 4-6a (R₆:RGD₂<-Pa) (0.5:9.5), the coassembled peptidesome of Example 4-6d (R₆:RGD₂<-Pa) (1:9) and the coassembled peptidesome of Example 4-6i (R₆:RGD₂<-Pa) (1.5:8.5), respectively, to SCC7 cells at 30 μM, the cells were incubated for 4 hours. Then, cell viability was measured by MTT assay.

In addition, the structure of the coassembled peptidesome of Example 4-6d (R₆:RGD₂<-Pa) (1:9) was investigated by TEM and AFM imaging.

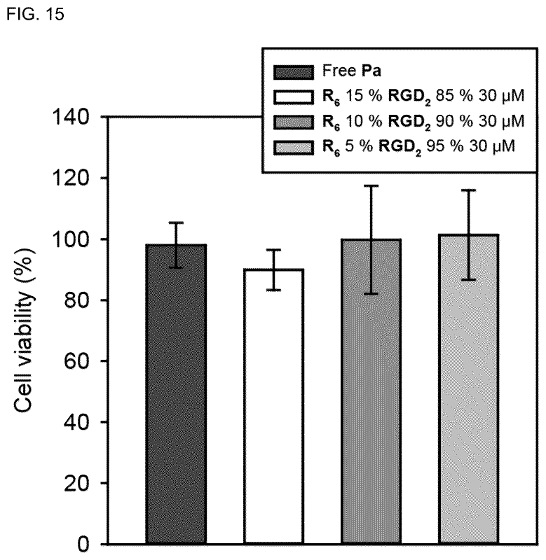

FIG. 15 shows a result of analyzing cell viability for the coassembled peptidesome of Example 4-6a (R₆:RGD₂<-Pa) (0.5:9.5), the coassembled peptidesome of Example 4-6d (R₆:RGD₂<-Pa) (1:9) and the coassembled peptidesome of Example 4-6i (R₆:RGD₂<-Pa) (1.5:8.5).

As seen from FIG. 15, all the coassembled peptidesomes of Example 4-6a, Example 4-6d and Example 4-6i exhibited cell viability of 90-100%. The coassembled peptidesome of Example 4-6d (R₆:RGD₂<-Pa) (1:9) showed the highest cell viability of 100%.

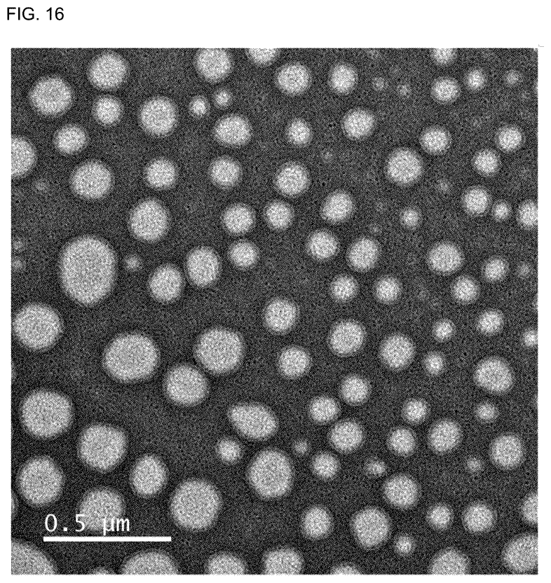

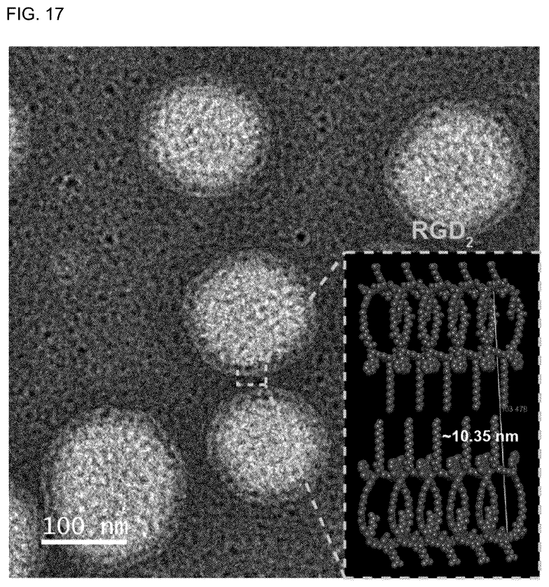

FIG. 16 and FIG. 17 are the TEM images of the coassembled peptidesome of Example 4-6d (R₆:RGD₂<-Pa) (1:9). FIG. 17 is an enlarged image of FIG. 16.

As shown in FIG. 16 and FIG. 17, it was confirmed that the morphology of the coassembled peptidesome of Example 4-6d (R₆:RGD₂<-Pa) (1:9) maintains the vesicular structure having a shell with a bilayer structure even after the coassembly.

In particular, as seen from FIG. 17, the coassembled peptidesome of Example 4-6d (R₆:RGD₂<-Pa) (1:9) had an average diameter of 149.6±70.6 nm and the thickness of the shell layer was 10.86±2.1 nm, which matched well with the schematic structure of the peptidesome calculated from the cyclic peptide (˜10.35 nm).

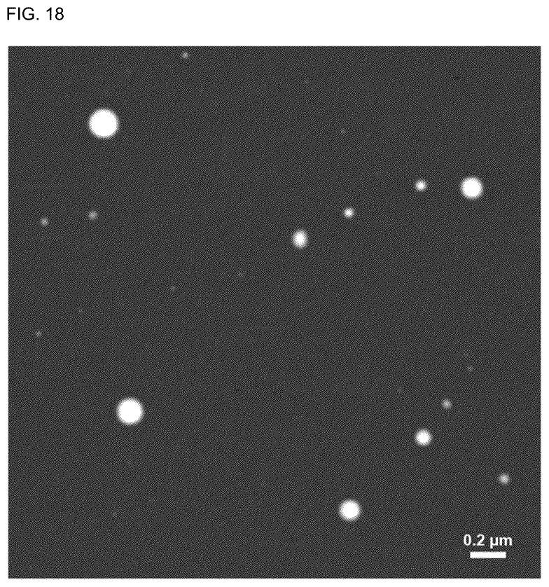

FIG. 18 is the AFM image of the coassembled peptidesome of Example 4-6d (R₆:RGD₂<-Pa) (1:9). It can be seen that the structure of the coassembled peptidesome of Example 4-6d (R₆:RGD₂<-Pa) (1:9) was not changed by the Pa encapsulation. In addition, the size of the coassembled peptidesome of Example 4-6d (R₆:RGD₂<-Pa) (1:9) was 113±68.5 nm.

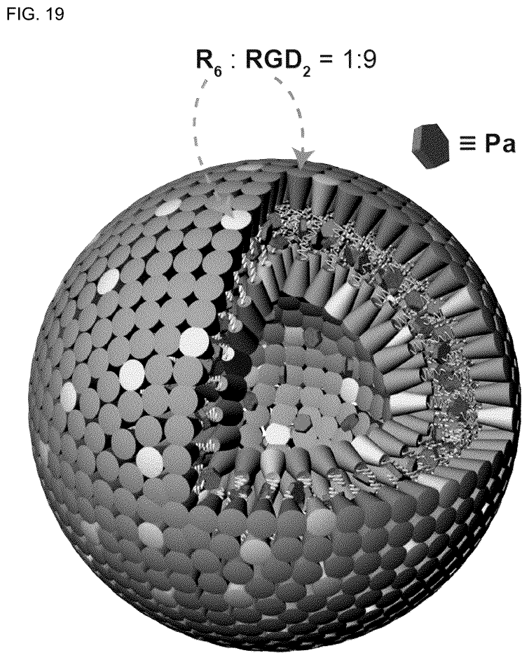

The structure of the coassembled peptidesome (R₆:RGD₂<-Pa) (1:9) determined from the experimental result is schematically shown in FIG. 19.

### Test Example 10. Intracellular Delivery Efficiency of Coassembled Peptidesome (R₆:RGD₂<-Pa)

After adding the coassembled peptidesome of Example 4-6d (R₆:RGD₂<-Pa) (1:9) to 0.5 mL of Hela cells at 30 μM, the cells were cultured for 4 hours. The cells were counted on a Lab-Tek chambered coverglass, stained with LysoTracker, and then imaged by CLSM (confocal laser scanning microscopy).

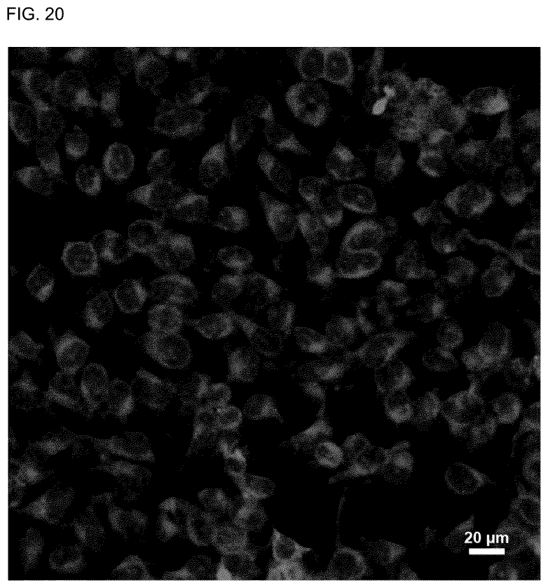

FIG. 20 shows a result of treating the HeLa cells with the coassembled peptidesome of Example 4-6d (R₆:RGD₂<-Pa) (1:9) and then investigating fluorescence from Pa (red) by CLSM (confocal laser scanning microscopy). FIGS. 21A and 21B show results of treating the HeLa cells with the coassembled peptidesome of Example 4-6d (R₆:RGD₂<-Pa) (1:9) and then investigating fluorescence from Pa (red) and LysoTracker (green). A fluorescence image (FIG. 21A) and an enlarged image (FIG. 21B) are shown.

As shown in FIG. 20 and FIGS. 21A to 21B, the coassembled peptidesome of Example 4-6d (R₆:RGD₂<-Pa) (1:9) is very effective for intracellular delivery of Pa. The minimal colocalization with LysoTracker indicates that the coassembled peptidesome (R₆:RGD₂<-Pa) enters the cell via the direct penetration mechanism and therefore is not trapped in endosomes. That is to say, it can be seen that the coassembled peptidesome (R₆:RGD₂<-Pa) according to the present disclosure is nontoxic while mediating effective delivery into the cytosol and nucleus. Moreover, the peptidesome containing the RGD sequence has cancer-targeting capability via RGD-integrin interaction and superior in-vivo performance due to the zwitterionic character of RGD.

### Test Example 11. Prevention of Aggregate Formation Under In-Vivo Conditions

In order to verify the superior intracellular delivery efficiency and in-vivo stability without aggregation of the peptidesome according to the present disclosure, photodynamic effect for cancer cells was evaluated.

SSC7 (squamous cell carcinoma 7) cells were treated with free Pa or the coassembled peptidesome of Example 4-6d (R₆:RGD₂<-Pa) (1:9) at 30 μM for 24 hours, followed by infrared (IR) laser (671 nm) irradiation at 1.59 J/cm². Cytotoxicity as a measure of the photodynamic killing of the cancer cells was determined by MTT assay.

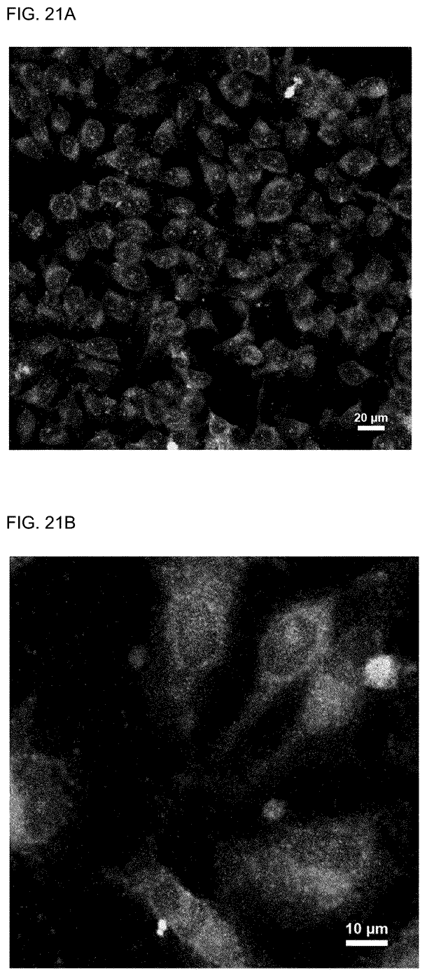

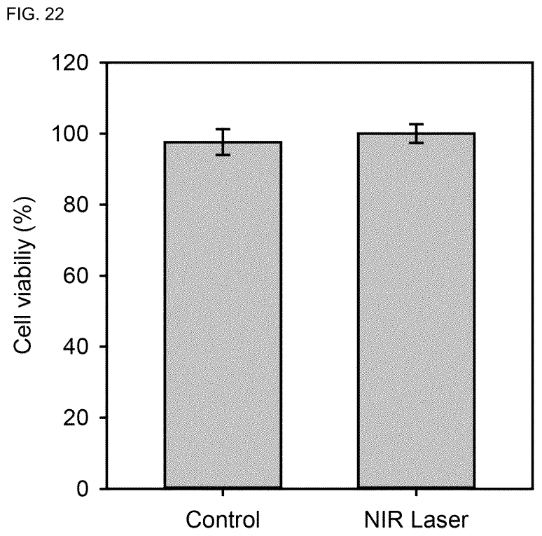

FIG. 22 shows the result of irradiating laser to the SCC7 cells and analyzing the viability of the cells by MTT. The viability of the cells irradiated with the laser only was about 97.6%, which means that the laser did not significantly affect the cell survival.

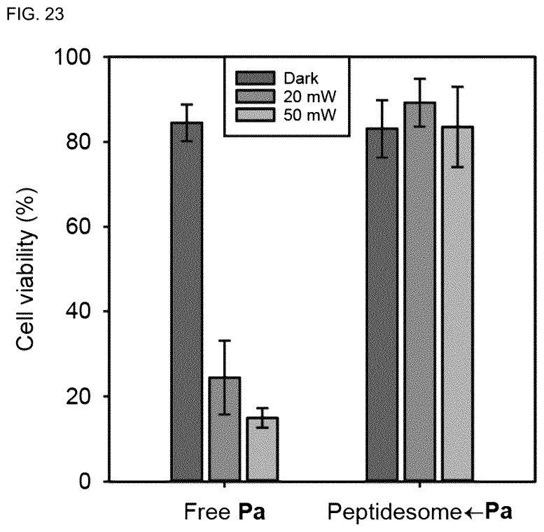

FIG. 23 shows the result of treating the SCC7 cells with free Pa and the coassembled peptidesome of Example 4-6d (R₆:RGD₂<-Pa) (1:9) and measuring cell viability after laser irradiation for analysis of photodynamic anticancer efficacy. As shown in FIG. 23, the cells treated with the coassembled peptidesome of Example 4-6d (R₆:RGD₂<-Pa) (1:9) showed no PDT effect, whereas the cells treated with free Pa showed dose-dependent PDT effect.

The following experiment was performed to probe the reason why the coassembled peptidesome of Example 4-6d (R₆:RGD₂<-Pa) (1:9) did not show photodynamic anticancer efficacy. First, after adding 30 μM free Pa or the coassembled peptidesome of Example 4-6d (R₆:RGD₂<-Pa) (1:9) to an RPMI 1640 medium containing 10% fetal bovine serum, followed by IR laser irradiation (10 seconds, 30 seconds), the content of singlet oxygen (SO) was quantified by detection of ROS generation.

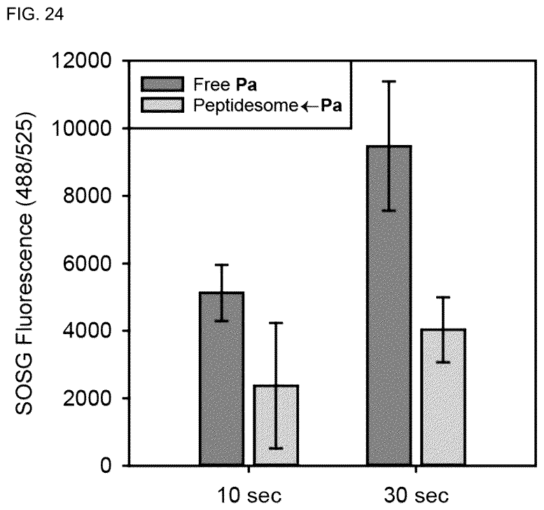

FIG. 24 shows the result of quantifying the singlet oxygen of free Pa and a coassembled peptidesome of Example 4-6d (R₆:RGD₂<-Pa) (1:9) with SOSG (Singlet Oxygen Sensor Green).

As shown in FIG. 24, the content of singlet oxygen (SO) was higher for the free Pa than the coassembled peptidesome of Example 4-6d (R₆:RGD₂<-Pa) (1:9).

The structure of the coassembled peptidesome of Example 4-6d (R₆:RGD₂<-Pa) (1:9) was analyzed under a physiological condition. For this, after adding 30 μM of the coassembled peptidesome of Example 4-6d (R₆:RGD₂<-Pa) (1:9) to an RPMI 1640 medium not containing 10% fetal bovine serum, followed by IR laser irradiation for 30 seconds, imaging was performed by AFM.

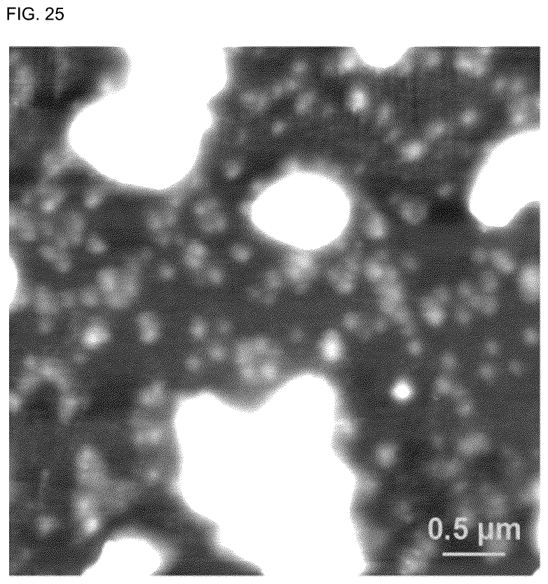

FIG. 25 shows the AFM image obtained after adding the coassembled peptidesome of Example 4-6d (R₆:RGD₂<-Pa) (1:9) to a serum-free RPMI 1640 medium.

It was confirmed that discrete (not aggregated) peptidesomes and aggregated peptidesomes coexist under the physiological condition. The aggregation of the peptidesome was more severe in the serum-containing cell culture medium. The nonspecific aggregation of the peptidesome according to the present disclosure under the physiological condition is responsible for the reduced SO generation and the lack of PDT effect. Therefore, it is necessary to reduce the aggregation of the peptidesome.

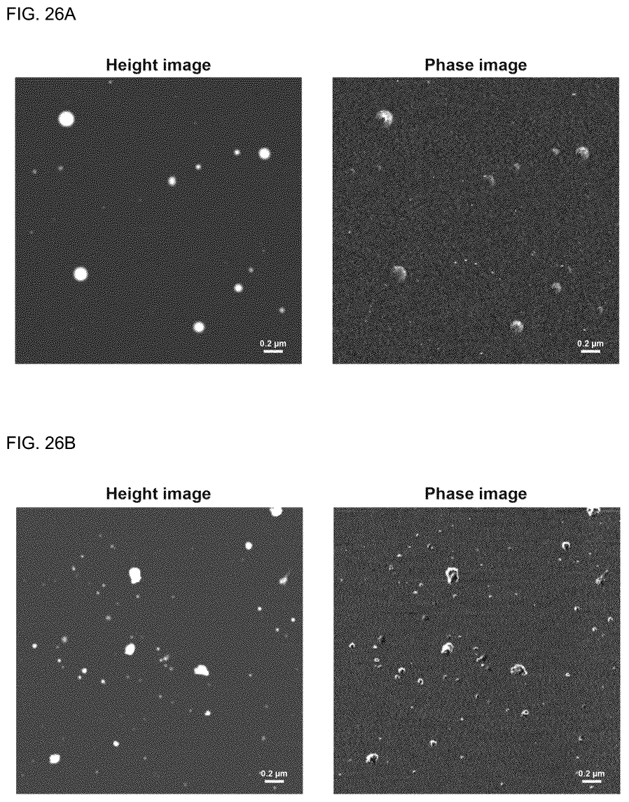

FIGS. 26A and 26B show the AFM images of the coassembled peptidesome of Example 4-6d (R₆:RGD₂<-Pa) (1:9). FIG. 26A is an image obtained before NIR irradiation and FIG. 26B is an image obtained after NIR irradiation.

As a result of analyzing the peptidesome morphology before and after the NIR irradiation, the size of the coassembled peptidesome of Example 4-6d (R₆:RGD₂<-Pa) (1:9) was changed before and after the NIR irradiation but there was no significant difference in the overall size distribution or vesicular structure.

### Test Example 5. DLS of Coassembled Peptidesome of Example 4-6d (R₆:RGD₂<-Pa) (1:9)

The average diameter of the coassembled peptidesome of Example 4-6d (R₆:RGD₂<-Pa) (1:9) was analyzed for different solution conditions. DLS analysis was conducted by adding the coassembled peptidesome of Example 4-6d (R₆:RGD₂<-Pa) (1:9) to solutions containing a physiologically relevant amount of salt and buffer (e.g., PBS), serum-free medium and 0.9% saline. The solution conditions and the measurement results are shown in Table 2.

TABLE 2

| Solution conditions | Diameter (Dₕ)^{a} | |
|---|---|---|
| Distilled water (DW) | 104 | nm |
| Phosphate-buffered saline (PBS) | ca. 400-700 | nm |
| Serum-free medium | ca. 500-1600 | nm |
| 5 wt % glucose | 117 | nm |
| 5 wt % glucose + 0.9 wt % saline | 410 | nm |
| 5 wt % glucose + 20 wt % glycerol | 100 | nm |
| 5 wt % glucose + 20 wt % glycerol + 0.9 wt % saline | 310 | nm |

^{a}Dₕ was measured using DLS.

As shown in Table 2, the severe aggregation of the coassembled peptidesome of Example 4-6d (R₆:RGD₂<-Pa) (1:9) was reconfirmed. It was confirmed that the aggregation became more severe in the presence of serum. In addition, it was confirmed that salt ions also induce the formation of large aggregates by decreasing colloidal stability. That is to say, the coassembled peptidesome of Example 4-6d (R₆:RGD₂<-Pa) (1:9) according to the present disclosure forms elongated superstructures as the hydrophobic strength is increased under high ionic strength conditions.

As a result of analyzing the formation of aggregates by the coassembled peptidesome of Example 4-6d (R₆:RGD₂<-Pa) (1:9) under various physiological conditions, an optimal condition in which the average diameter can be maintained at <150 nm, specifically <100 nm, was determined.

First, it was confirmed that the coassembled peptidesome of Example 4-6d (R₆:RGD₂<-Pa) (1:9) maintains an average diameter of 104 nm in distilled water, and 117 nm in a 5 wt % glucose aqueous solution. Glucose is a biocompatible molecule and has been widely used as an infusion.

Then, it was confirmed that the coassembled peptidesome of Example 4-6d (R₆:RGD₂<-Pa) (1:9) has an average diameter of 100 nm in a 5 wt % glucose+20 wt % glycerol aqueous solution. That is to say, it was confirmed that it is the most preferable to use a polyol for structural stabilization of the peptidesome according to the present disclosure.

It is thought that the polyol increases the colloidal stability of the peptidesome according to the present disclosure and helps maintain the in-vivo osmolarity and tonicity of the peptidesome injection. It was confirmed that the peptidesome according to the present disclosure does not form aggregates in a solution containing one or more selected from a group consisting of glucose, a polyol and distilled water, and has an average diameter smaller than 150 nm, 140 nm, 130 nm or 120 nm. More specifically, the solution may be a mixture of glucose, a polyol and distilled water, further more specifically a solution containing 1-10 wt % glucose, 10-30 wt % of a polyol distilled water as the balance, a solution containing 2-7 wt % of glucose, 15-25 wt % of a polyol and distilled water as the balance, or a solution containing 4-6 wt % of glucose, 17-22 wt % of a polyol and distilled water as the balance. When the above ranges are satisfied, the average diameter of the peptidesome may be maintained at <100 nm.

The polyol is not specially limited as long as it has superior biocompatibility. It may be specifically one or more polyol selected from a group consisting of ethylene glycol, propanediol, butanediol, pentanediol, hexanediol, glycerol and polyethylene glycol, more specifically one or more polyol selected from a group consisting of ethylene glycol, glycerol and polyethylene glycol, most specifically glycerol.

When the peptidesome according to the present disclosure is stored in a mixture of 5 wt % of glucose, 20 wt % of glycerol and distilled water as the balance (hereinafter, also referred to as ‘G&G’), aggregation can be prevented and an average diameter of smaller than 100 nm can be maintained.

As described above, it was confirmed the peptidesome stably maintains a small size without aggregation in a mixture of 5 wt % of glucose, 20 wt % of glycerol and distilled water as the balance (G&G).

After adding 30 μM of the coassembled peptidesome of Example 4-6d (R₆:RGD₂<-Pa) (1:9) to an RPMI 1640 medium containing 10% (v/v) fetal bovine serum, average diameter was analyzed by DLS 1 hour later.

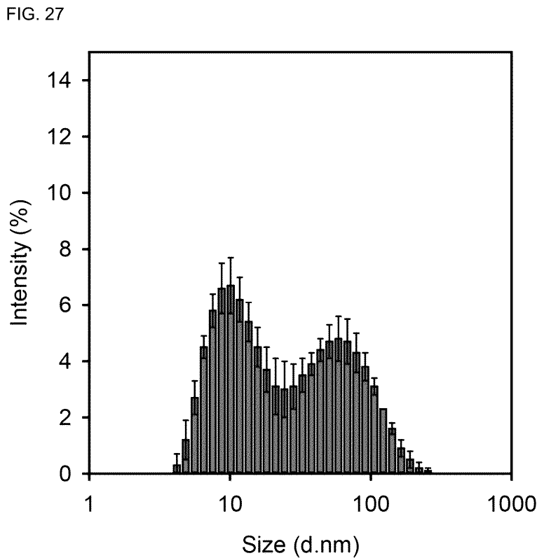

FIG. 27 shows the size distribution of the coassembled peptidesome of Example 4-6d (R₆:RGD₂<-Pa) (1:9) in the presence of serum proteins obtained by measuring average diameter. As seen from FIG. 27, the coassembled peptidesome of Example 4-6d (R₆:RGD₂<-Pa) (1:9) maintained the size of 100 nm without aggregation even in the presence of serum proteins. In FIG. 27, the peak at 10 nm is that of the serum proteins.

### Test Example 6. Analysis of Intracellular Delivery Efficiency

SCC7 cells were seeded in a 24-well plate at a density of 5×10⁴ and incubated overnight. Then, 200 mL of the sample was added to 800 mL of a culture medium. The cells were treated with the sample for 4 hours, followed by removal of the sample and washing. For cell imaging, the cell nuclei were stained with Hoechst 33342. The uptake of materials by the SCC7 cells was analyzed based on the intrinsic fluorescence of Pa via CLSM (LSM700, Carl Zeiss, Germany) and flow cytometry (FACS Canto II, BD Biosciences, Bedford, MA, USA).

As the samples, 2 μg/mL of free Pa, 2 μg/mL of the coassembled peptidesome of Example 4-6d (R₆:RGD₂<-Pa) (1:9) (G&G) and 2 μg/mL of the coassembled peptidesome of Example 4-6d (R₆:RGD₂<-Pa) (1:9) (DW) were used. DW means that distilled water was used instead of the G&G solution.

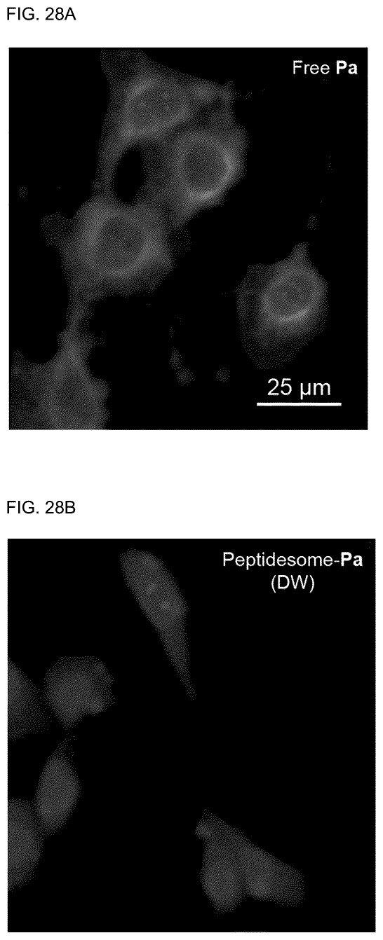

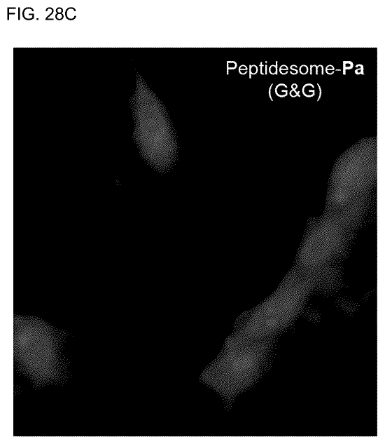

FIGS. 28A to 28C show the CLSM images of the SCC7 cells treated respectively with free Pa (FIG. 28A), the coassembled peptidesome of Example 4-6d (R₆:RGD₂<-Pa) (1:9) (DW) (FIG. 28B) and the coassembled peptidesome of Example 4-6d (R₆:RGD₂<-Pa) (1:9) (G&G) (FIG. 28C). In FIGS. 28A to 28C, red color indicates Pa and blue color indicates nucleus.

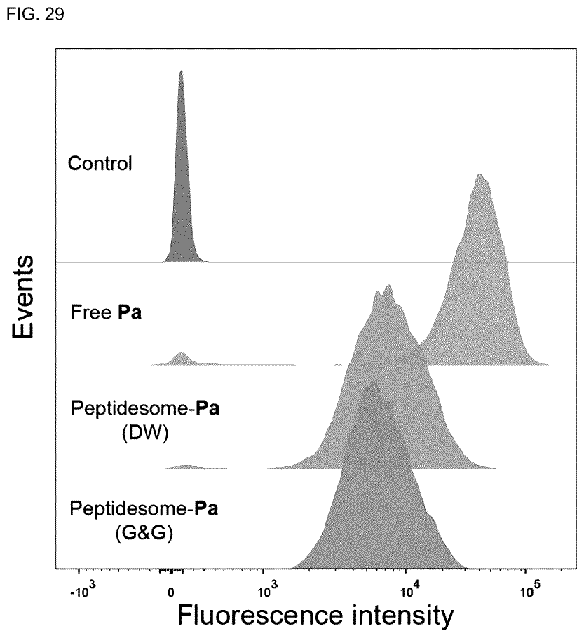

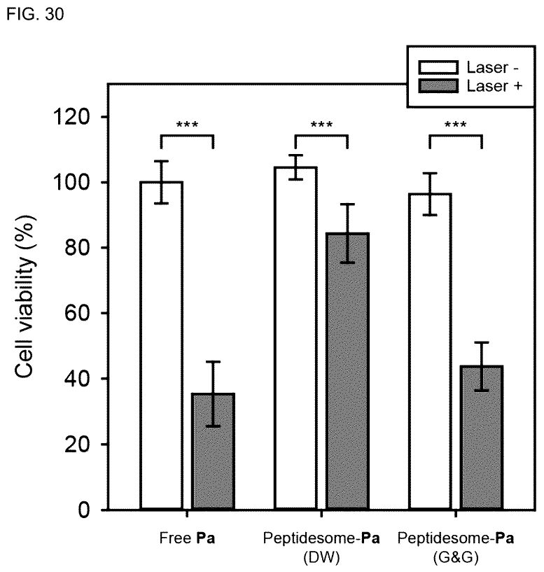

FIG. 29 shows a result of analyzing the SCC7 cells treated respectively with free Pa, the coassembled peptidesome of Example 4-6d (R₆:RGD₂<-Pa) (1:9) (DW) and the coassembled peptidesome of Example 4-6d (R₆:RGD₂<-Pa) (1:9) (G&G) by flow cytometry (FACS).

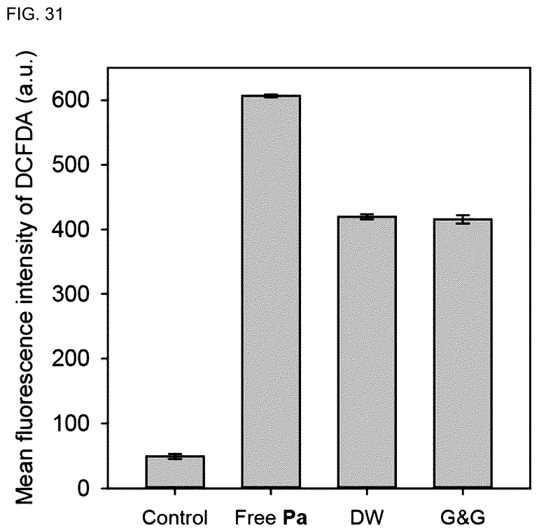

FIG. 30 shows a result of analyzing the viability of the SCC7 cells treated with free Pa, the coassembled peptidesome of Example 4-6d (R₆:RGD₂<-Pa) (1:9) (DW) and the coassembled peptidesome of Example 4-6d (R₆:RGD₂<-Pa) (1:9) (G&G), respectively.

Error bars represent mean±standard deviation (n=3). Statistical significance was tested by two-sample Student's t-test and p<0.005 was regarded as significant (*** p<0.001).

As shown in FIGS. 28A to 28C and FIG. 29, the cell internalization efficiency of the free Pa was 5-fold higher than the coassembled peptidesome of Example 4-6d (R₆:RGD₂<-Pa) (1:9). It is thought that the lipophilic free Pa accounts for the high intracellular delivery efficiency.

Although it was confirmed that the degree of aggregate formation by the coassembled peptidesome of Example 4-6d (R₆:RGD₂<-Pa) (1:9) varies depending on solutions, no significant difference was observed in the actual cell internalization efficiency. This means that the aggregation of the peptidesome is not an important factor in the intracellular delivery efficiency of the peptidesome. It was confirmed that the peptidesome according to the present disclosure has superior intracellular delivery efficiency regardless of aggregation.

As seen from FIG. 30, the PDT effect of the coassembled peptidesome of Example 4-6d (R₆:RGD₂<-Pa) (1:9) (G&G) was 2-fold higher than the coassembled peptidesome of Example 4-6d (R₆:RGD₂<-Pa) (1:9) (DW). In other words, even if the peptidesome according to the present disclosure shows superior intracellular delivery efficiency regardless of aggregation, the aggregation does influence the anticancer effect in cells. The anticancer effect of the coassembled peptidesome of Example 4-6d (R₆:RGD₂<-Pa) (1:9) (G&G) was 2-fold higher than the coassembled peptidesome of Example 4-6d (R₆:RGD₂<-Pa) (1:9) (DW).

FIG. 31 shows a result of analyzing ROS production from SSC7 cells treated with free Pa, the coassembled peptidesome of Example 4-6d (R₆:RGD₂<-Pa) (1:9) (DW) and the coassembled peptidesome of Example 4-6d (R₆:RGD₂<-Pa) (1:9) (G&G) by DCFDA. Error bars represent mean±standard deviation (n=3).

As shown in FIG. 31, as a result of analyzing PDT-induced ROS generation in vitro using DCFDA (2′,7′-dichlorofluorescin diacetate) and an ROS detection agent, DCFDA fluorescence was significantly increased under laser irradiation in the SCC7 cells treated with the peptidesome (R₆:RGD₂<-Pa) (1:9) (G&G) as compared to a control group (untreated SSC7 cells). This suggests that ROS generation is increased by the photodynamic effect of the peptidesome (R₆:RGD₂<-Pa) (1:9) (G&G) and, thus, cancer cells are killed.

### Test Example 7. Analysis of Anticancer Effect of Peptidesome In Vivo 1

The anticancer effect of the peptidesome according to the present disclosure in vivo was analyzed. For this, the in-vivo antitumor efficacy was analyzed using a xenograft mouse model bearing SCC7 cell-derived cancer.

Squamous cell carcinoma 7 (SSC7) cells were cultured in an RPMI 1640 medium containing 10% FBS, 1% L-glutamine, 1% penicillin, streptomycin, etc. in a 37° C. incubator maintained at 5% carbon dioxide. The cultured cells were detached from the bottom of the flask using trypsin/EDTA and cell viability was evaluated by trypan blue exclusion assay. The final concentration of the culture was set to 1×10⁶ cells/mL.

7- to 8-week-old female or male xenograft mice (OrientBio, Korea) were used as experimental animals. The mice were acclimatized for 2 weeks before experiment. The mice were kept in a breeding room maintained at 22±2° C. and 40-60% humidity during the experiment and were allowed free access to feed. The light and dark cycles were adjusted at 12-hour intervals. After the acclimatization for a week, the prepared SSC7 cells were injected into the left thigh, with 1×10⁶ cells per mouse. Then, the mice were bred until the tumor volume reached 50 mm³ to prepare a cancer animal model.

The cancer animal model was randomly grouped, with 3 mice per group. Test groups were a free Pa 2 mg/kg comparison group (free Pa, n=3), a coassembled peptidesome of Example 4-6d (R₆:RGD₂<-Pa) (1:9) (DW) 2 mg/kg administration group (peptidesome-Pa(saline), n=3) and a coassembled peptidesome of Example 4-6d (R₆:RGD₂<-Pa) (1:9) (DW) 2 mg/kg administration group (peptidesome-Pa(G&G), n=3). The samples were injected via the tail vein of the cancer animal model. 3 hours after the sample injection, NIR laser (671 nm) was irradiated for 15 minutes with a power of 0.53 W/cm². The same sample injection and NIR laser irradiation procedures were repeated on the next day.

After the injection of the sample to each group, whole-body NIR fluorescence images were analyzed using an IVIS Lumina XRMS system at 3 hours, 6 hours, 12 hours and 24 hours, and blood taken at each time point was observed with a fluorescence microscope. At the last time point, the animal model was euthanized and major organs (heart, lung, liver, spleen and kidney) and cancer tissues were dissected. The organs and cancer tissues were fixed, prepared into cryosections and observed with a fluorescence microscope.

This research was approved by the Yonsei University Animal Care and Use Committee and all experiments were conducted in accordance with the regulations of the Yonsei University Animal Care and Use Committee.

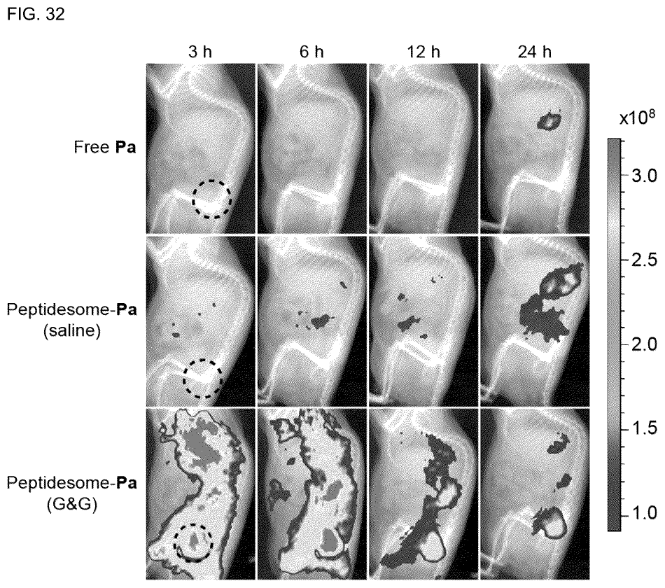

FIG. 32 shows the time-dependent whole-body NIR fluorescence images of the free Pa comparison group, the coassembled peptidesome of Example 4-6d (R₆:RGD₂<-Pa) (1:9) (DW) administration group and the coassembled peptidesome of Example 4-6d (R₆:RGD₂<-Pa) (1:9) (DW) administration group. The black dotted circles indicate tumor regions.

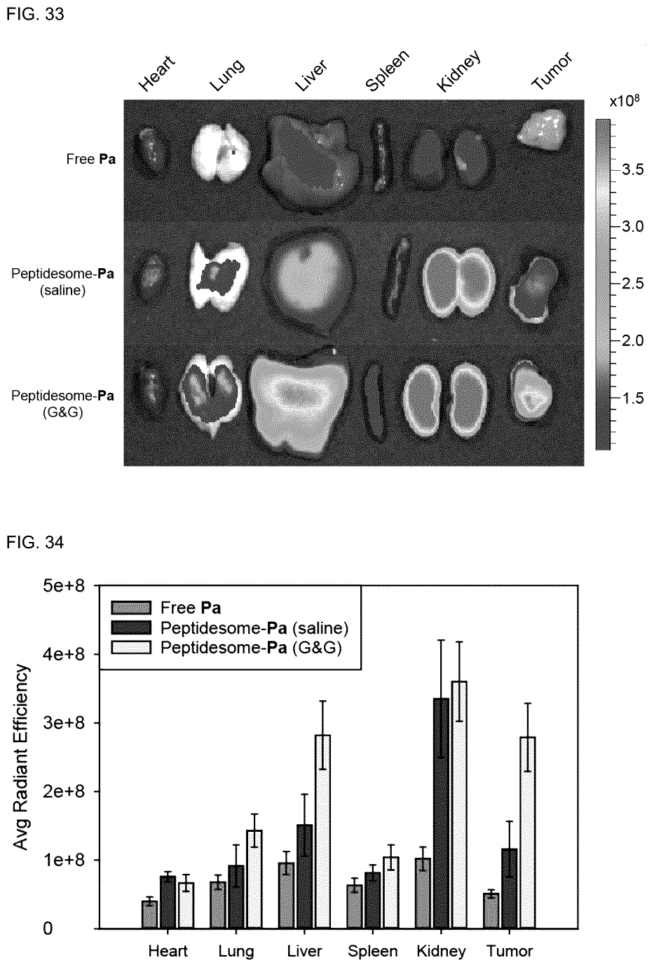

FIG. 33 shows the fluorescence microscopic images of the organs isolated from the free Pa comparison group, the coassembled peptidesome of Example 4-6d (R₆:RGD₂<-Pa) (1:9) (DW) administration group and the coassembled peptidesome of Example 4-6d (R₆:RGD₂<-Pa) (1:9) (DW) administration group.

FIG. 34 shows fluorescence intensities quantified from the data of FIG. 33. Error bars represent mean±standard deviation (n=3).

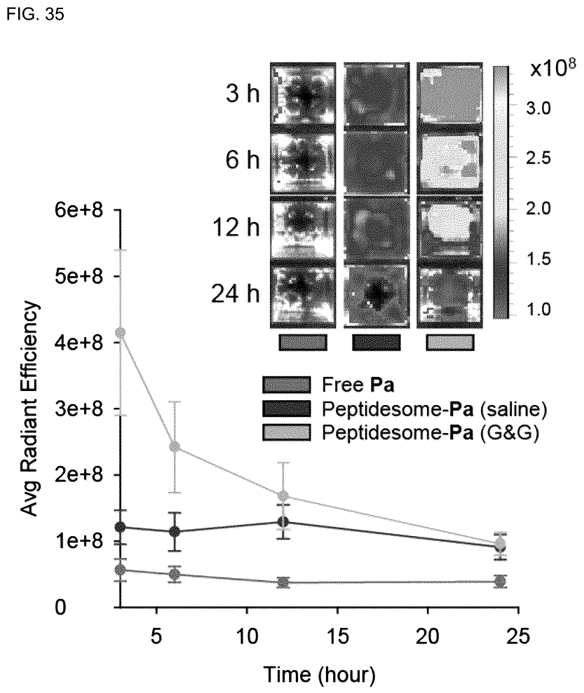

FIG. 35 shows the fluorescence microscopic images of the blood taken from the free Pa comparison group, the coassembled peptidesome of Example 4-6d (R₆:RGD₂<-Pa) (1:9) (DW) administration group and the coassembled peptidesome of Example 4-6d (R₆:RGD₂<-Pa) (1:9) (DW) administration group (top) and a quantification result thereof (bottom) (n=3).

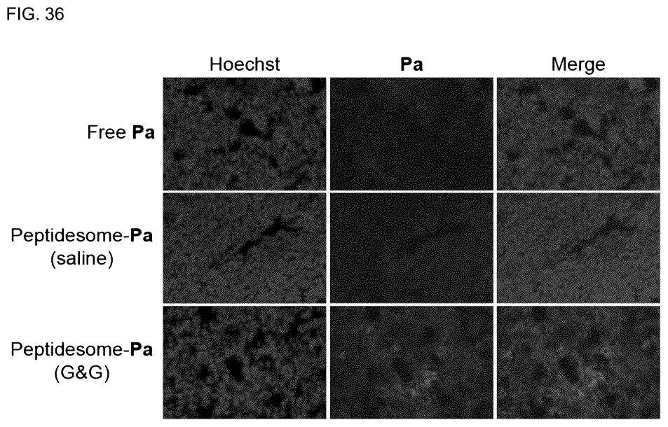

FIG. 36 shows the fluorescence microscopic images of the cryosection samples of cancer tissues taken from the free Pa comparison group, the coassembled peptidesome of Example 4-6d (R₆:RGD₂<-Pa) (1:9) (DW) administration group and the coassembled peptidesome of Example 4-6d (R₆:RGD₂<-Pa) (1:9) (DW) administration group.

It was investigated whether the drug (Pa) was accumulated at the tumor site through whole-body fluorescence images as shown in FIG. 32. Significantly more intense Pa fluorescence was observed for the coassembled peptidesome of Example 4-6d (R₆:RGD₂<-Pa) (1:9) (G&G) administration group as compared to the free Pa comparison group and the coassembled peptidesome of Example 4-6d (R₆:RGD₂<-Pa) (1:9) (DW) administration group. Nanoparticles were severely aggregated for the coassembled peptidesome of Example 4-6d (R₆:RGD₂<-Pa) (1:9) (DW) because the coassembled peptidesome of Example 4-6d (R₆:RGD₂<-Pa) (1:9) (DW) was diluted with 0.9% saline for intravenous injection.

As seen from FIGS. 33-35, the coassembled peptidesome of Example 4-6d (R₆:RGD₂<-Pa) (1:9) (G&G) showed the highest tumor accumulation efficiency as compared to other samples. The fluorescence of the coassembled peptidesome of Example 4-6d (R₆:RGD₂<-Pa) (1:9) (G&G) was also significantly higher in blood.

As seen from FIG. 36, the administration of the coassembled peptidesome of Example 4-6d (R₆:RGD₂<-Pa) (1:9) (G&G) resulted in very superior tumor accumulation efficiency and anticancer effect as compared to other therapies or samples. It can be seen that the coassembled peptidesome of Example 4-6d (R₆:RGD₂<-Pa) (1:9) (G&G) has significantly superior effect in targeting cancer due to the EPR (enhanced permeability and retention) effect and RGD-integrin interactions.

Furthermore, the peptidesome according to the present disclosure exhibits high tumor-specific targeting efficacy and significantly high drug accumulation efficiency in cancer cells since the aggregation of the peptidesome is suppressed and interrupted. It can be seen that it is the most preferable to use the G&G solution instead of distilled water.

### Test Example 8. Analysis of Anticancer Effect of Peptidesome In Vivo 2

The anticancer effect of the peptidesome according to the present disclosure in vivo was analyzed. For this, the in-vivo antitumor efficacy was analyzed using a xenograft mouse model bearing SCC7 cell-derived cancer.

Squamous cell carcinoma 7 (SSC7) cells were cultured in an RPMI 1640 medium containing 10% FBS, 1% L-glutamine, 1% penicillin, streptomycin, etc. in a 37° C. incubator maintained at 5% carbon dioxide. The cultured cells were detached from the bottom of the flask using trypsin/EDTA and cell viability was evaluated by trypan blue exclusion assay. The final concentration of the culture was set to 1×10⁶ cells/mL.

7- to 8-week-old female or male xenograft mice (OrientBio, Korea) were used as experimental animals. The mice were acclimatized for 2 weeks before experiment. The mice were kept in a breeding room maintained at 22±2° C. and 40-60% humidity during the experiment and were allowed free access to feed. The light and dark cycles were adjusted at 12-hour intervals. After the acclimatization for a week, the prepared SSC7 cells were injected into the left thigh, with 1×10⁶ cells per mouse. Then, the mice were bred until the tumor volume reached 50 mm³ to prepare a cancer animal model.

The cancer animal model was randomly grouped, with 4 mice per group. Test groups were a control group to which only 2 mg/kg saline was administered (saline, n=4), a free Pa 2 mg/kg comparison group (free Pa, n=4) and a coassembled peptidesome of Example 4-6d (R₆:RGD₂<-Pa) (1:9) (DW) 2 mg/kg administration group (peptidesome-Pa (G&G), n=4). The samples were injected via the tail vein of the cancer animal model. 3 hours after the sample injection, NIR laser (671 nm) was irradiated for 15 minutes with a power of 0.53 W/cm². The same sample injection and NIR laser irradiation procedures were repeated on the next day. The laser intensity used was set at a level that did not cause damage to the cancer cells.

After the injection of the sample to each group, tumor volume and body weight were recorded 2 days, 4 days, 6 days, 8 days, 10 days, 12 days and 14 days later. At the last time point, the animal model was euthanized and major organs (heart, lung, liver, spleen and kidney) and cancer tissues were dissected. The organs and cancer tissues were fixed, prepared into cryosections, stained with hematoxylin and eosin (H&E) and observed with a fluorescence microscope (Axiolmager A1, Zeiss, Germany).

This research was approved by the Yonsei University Animal Care and Use Committee and all experiments were conducted in accordance with the regulations of the Yonsei University Animal Care and Use Committee.

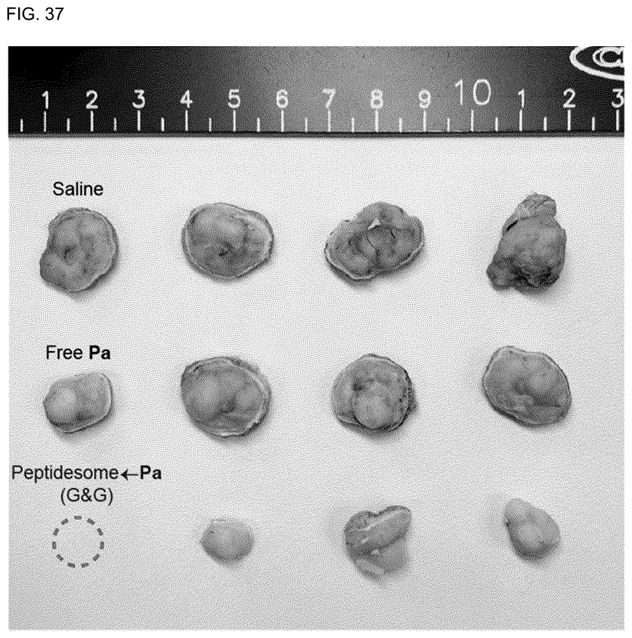

FIG. 37 shows the images of the cancer tissues isolated from the control group (saline), the free Pa comparison group and the coassembled peptidesome of Example 4-6d (R₆:RGD₂<-Pa) (1:9) (G&G) administration group after 14 days. The dotted circle (red) indicates complete regression of the tumor.

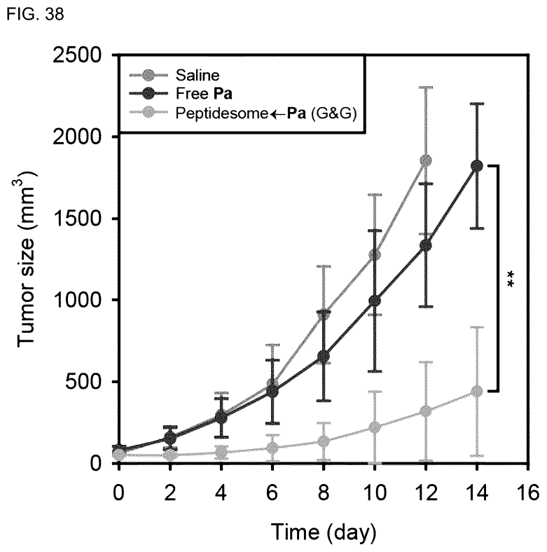

FIG. 38 shows a result of measuring the tumor size of the control group (saline), the free Pa comparison group and the coassembled peptidesome of Example 4-6d (R₆:RGD₂<-Pa) (1:9) (G&G) administration group at different times.

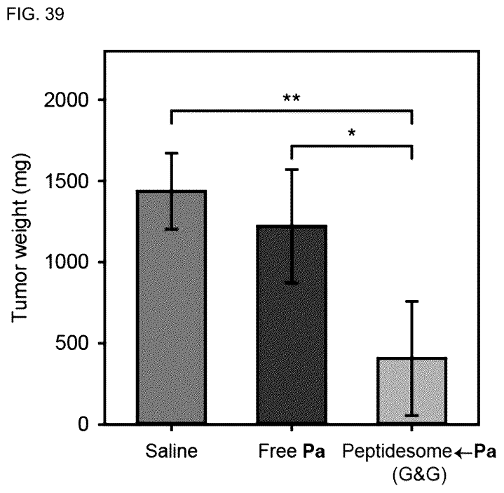

FIG. 39 shows a result of measuring the weight of the cancer tissues isolated from the control group (saline), the free Pa comparison group and the coassembled peptidesome of Example 4-6d (R₆:RGD₂<-Pa) (1:9) (G&G) administration group after 14 days.

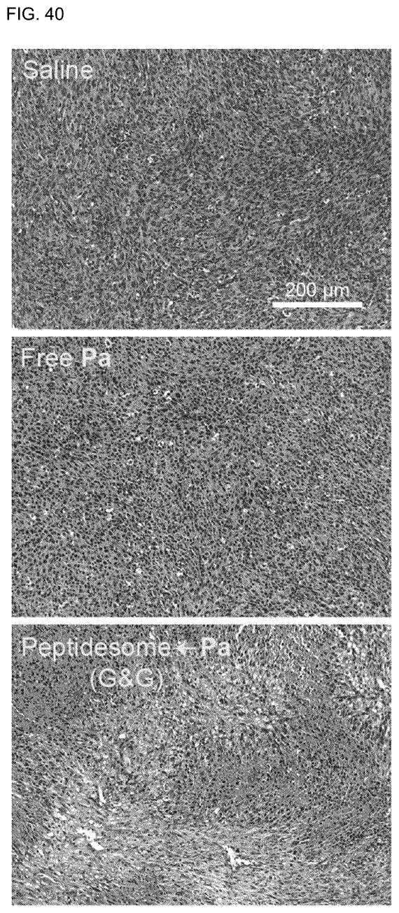

FIG. 40 shows the fluorescence microscopic images of the cancer tissues isolated from the control group (saline), the free Pa comparison group and the coassembled peptidesome of Example 4-6d (R₆:RGD₂<-Pa) (1:9) (G&G) administration group and stained with H&E after 14 days.

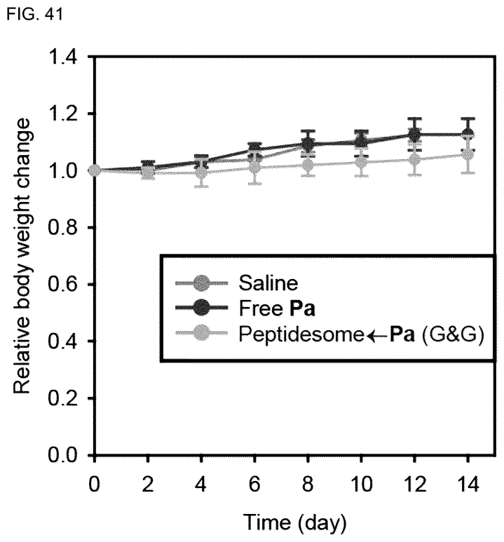

FIG. 41 shows a result of measuring the change in body weight of the control group (saline), the free Pa comparison group and the coassembled peptidesome of Example 4-6d (R₆:RGD₂<-Pa) (1:9) (G&G) administration group with time.

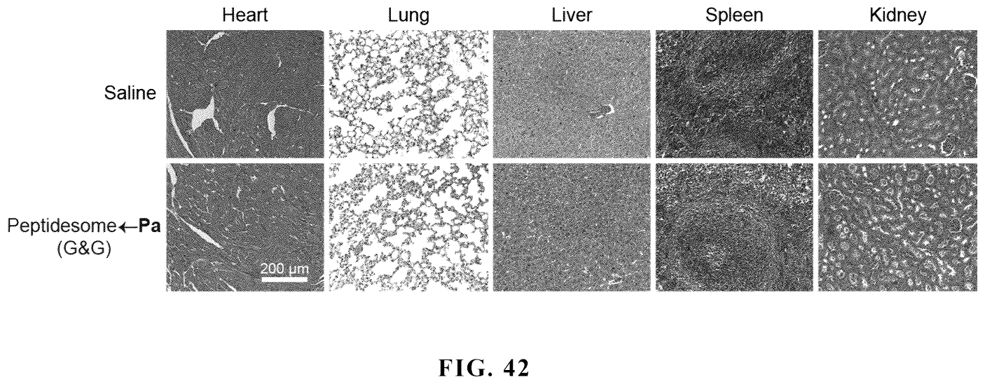

FIG. 42 shows the fluorescence microscopic images of the major organs (heart, lung, liver, spleen and kidney) isolated from the control group (saline), the free Pa comparison group and the coassembled peptidesome of Example 4-6d (R₆:RGD₂<-Pa) (1:9) (G&G) administration group and stained with H&E after 14 days.

In FIG. 38, FIG. 39 and FIG. 41, error bars represent mean±standard deviation (n=4). Statistical significance was tested by one-way ABOVA and post hoc Tukey's test in FIG. 38 and by two-sample Student's t-test in FIG. 39. * P<0.05, **P<0.01.

As seen from FIG. 37, tumor growth was significantly inhibited in the coassembled peptidesome of Example 4-6d (R₆:RGD₂<-Pa) (1:9) (G&G) as compared to other groups. Because the tumor grew too vigorously, the control group was euthanized on day 12 due to concerns about animal ethics.

As shown in FIG. 38, the tumor size of the animal model treated with the peptidesome<-Pa (G&G) on day 14 was 4.2 times smaller than that of the animal model treated with free Pa. This means that the peptidesome according to the present disclosure provides remarkably significant synergistic effect of 4.2 times or greater as compared to the treatment with the drug alone.

As seen from FIG. 39, at the end of the treatment for 14 days, the weight of the excised tumor for the free Pa group (1,222 mg) was approximately 3 times larger than that of the peptidesome<-Pa(G&G) group (406 mg).

As seen from FIG. 40, no significant change was observed for the body weight in all groups during the treatment period of 14 days. This indicates that the peptidesome<-Pa according to the present disclosure is safe with no side effect such as systemic toxicity.

As seen from FIG. 41, the H&E image of the cancer tissue from the coassembled peptidesome of Example 4-6d (R₆:RGD₂<-Pa) (1:9) (G&G) administration group showed a severely destroyed structure as compared the control group or the free Pa comparison group. It can be seen that the peptidesome according to the present disclosure (R₆:RGD₂<-Pa) increases anticancer effect by 2-4 times or more as compared to when the drug is administered alone.

As seen from FIG. 42, no significant damage was observed for the major organs (heart, lung, liver, spleen and kidney) of all the groups. This indicates that the peptidesome according to the present disclosure (R₆:RGD₂<-Pa) is safe with no side effect.

### Test Example 9. Analysis of In-Vivo Stability of Peptidesome

It was investigated whether the peptidesome according to the present disclosure (R₆:RGD₂<-Pa) can exist stably in the presence of a protease. First, the peptidesome of Example 3-4 (RGD₂) (50 μM, 300 μL) was mixed with trypsin from bovine pancreas (0.39 μg) in PBS buffer and the mixture was incubated at 37° C. Aliquots were taken at different times (0 minutes, 1 minute, 5 minutes, 15 minutes, 30 minutes, 1 hour, 2 hours, 6 hours, 12 hours, 24 hours) and analyzed by HPLC after adding 0.2% TFA (v/v). Before the HPLC analysis, acetonitrile was added to a final concentration of 50% (v/v) to disrupt molecular assembly. The reaction mixture was analyzed by reversed-phase HPLC using a C₄ column.

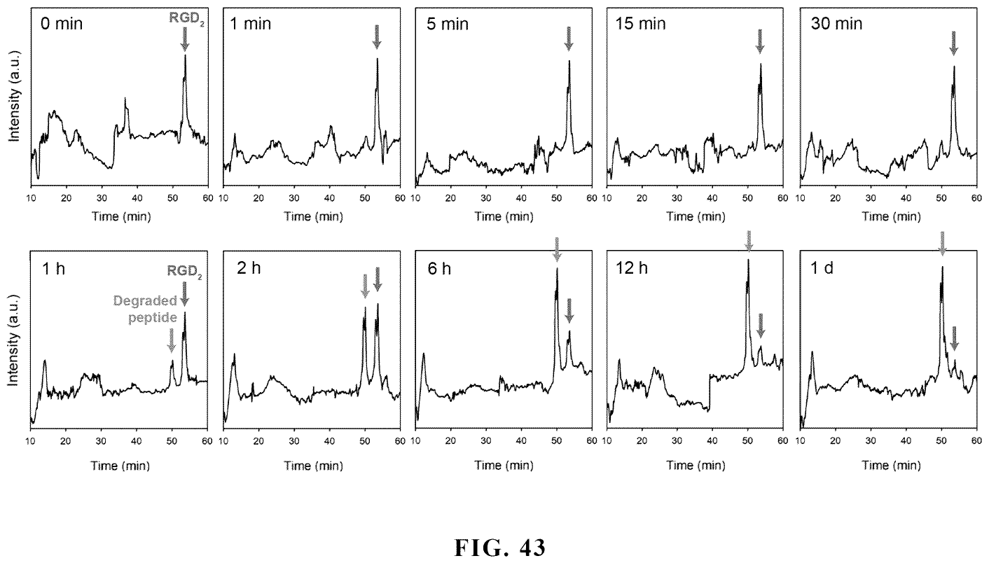

FIG. 43 shows the HPLC analysis result of the peptidesome of Example 3-4 (RGD₂) in the presence of the protease trypsin.

As seen from FIG. 43, the peptidesome of Example 3-4 (RGD₂) showed high resistance to proteolytic degradation under a physiological condition in vitro. Specifically, although a trace amount of the peptidesome of Example 3-4 (RGD₂) began to be degraded from 1 hour, it maintained its structure without complete degradation until 6 hours. Other peptidesomes also showed superior stability against the protease.

The peptidesome according to the present disclosure has superior stability against proteolytic degradation since it forms a tight molecular assembly even though it is formed from a low-molecular-weight cyclic peptide containing two or more arginine residues. In general, one of the most fatal weakness of drug carriers, particularly drug carriers prepared from peptides, is rapid proteolytic degradation in vivo. Since the peptidesome according to the present disclosure is very stable against proteolytic degradation, it can effectively deliver a drug.

What is claimed is:

1. A cyclic peptide comprising:
(a) a hydrophilic peptide consisting of 2 to 12 L- or D-arginine residues; and
(b) a hydrophobic peptide represented by General Formula 1,
wherein the (a) and the (b) are linked by a linker:
Xaa1-Lys-Xaa2 [General Formula 1]
wherein
each of Xaa1 and Xaa2 is independently tryptophan (W) or phenylalanine (F).

2. The cyclic peptide according to claim 1, wherein, in General Formula 1, Xaa1 is bonded to the ε-amino group of the lysine residue (Lys).

3. The cyclic peptide according to claim 1, wherein the (a) is a hydrophilic peptide consisting of 2 to 10 L- or D-arginine residues.

4. The cyclic peptide according to claim 1, wherein, in the hydrophobic peptide (b), a hydrophobic ligand or a hydrophobic drug is bonded to the α-amino group of the lysine residue.

5. The cyclic peptide according to claim 4, wherein the hydrophobic ligand is any one selected from a C₈-C₂₄ fatty acid.

6. The cyclic peptide according to claim 5, wherein the fatty acid is any one selected from a group consisting of oleic acid, lauric acid, palmitic acid, linoleic acid and stearic acid.

7. The cyclic peptide according to claim 4, wherein the hydrophobic drug is any anticancer agent selected from doxorubicin, paclitaxel, cisplatin, sirolimus and etoposide or any photosensitizer selected from a phthalocyanine-based compound, a porphyrin-based compound, a fluorescein-based compound and a chlorin-based compound.

8. The cyclic peptide according to claim 1, wherein the linker is any one selected from a linker peptide, Ebes and an oligoethylene glycol (OEG) represented by SEQ ID NOS 12-19.

9. The cyclic peptide according to claim 1, wherein the hydrophilic peptide (a) has a sequence represented by any one selected from SEQ ID NOS 1-7.

10. The cyclic peptide according to claim 1, wherein the cyclic peptide is any one selected from the compounds represented by Chemical Formulas 1-5:

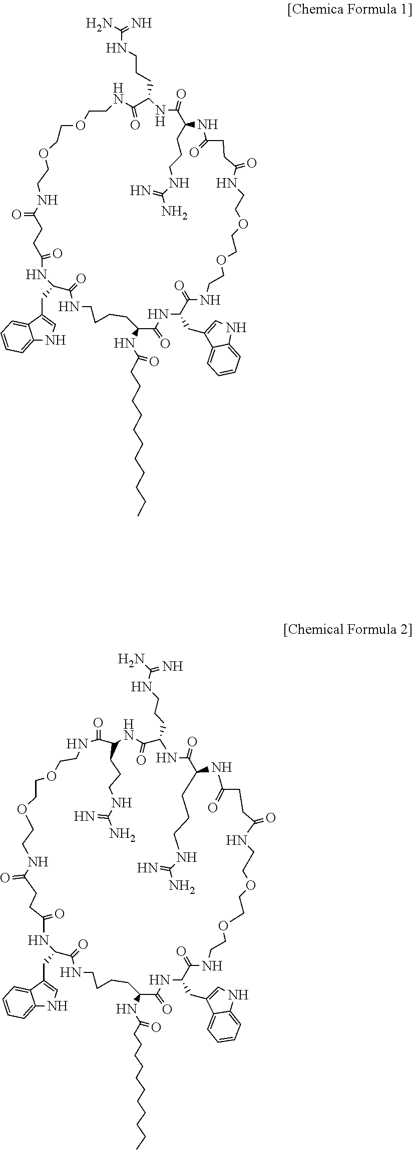

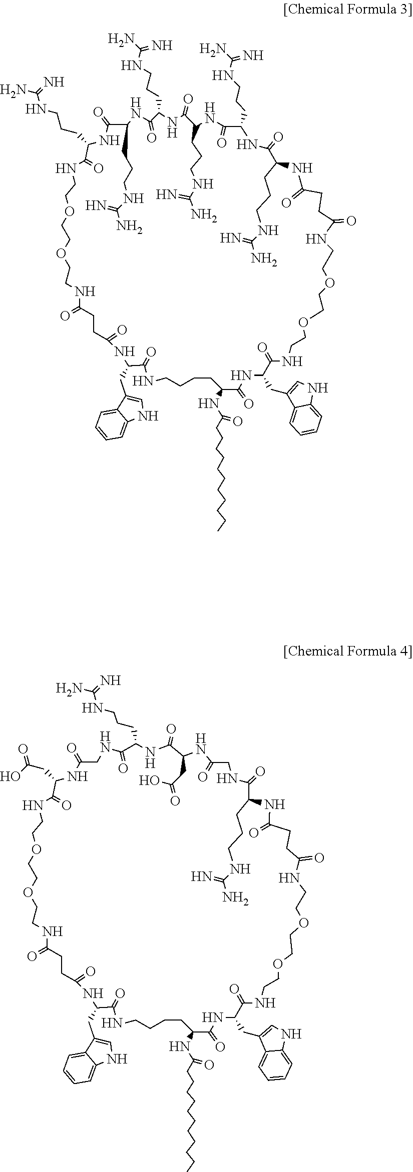

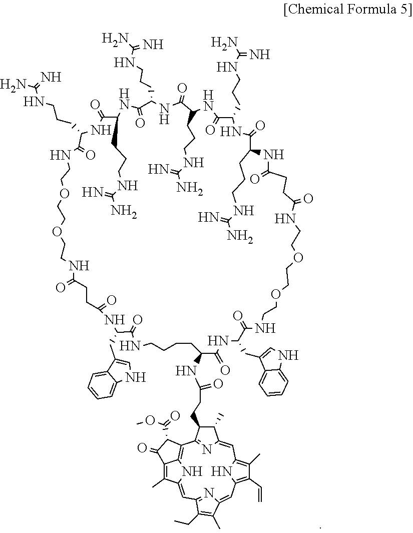

11. The cyclic peptide according to claim 1, wherein the cyclic peptide self-assembles into a vesicular peptidesome in a solution.

12. A spherical peptidesome having a vesicular structure, which is formed as at least one cyclic peptide according to claim 1 self-assembles in a liquid.

13. The peptidesome according to claim 12, wherein the peptidesome consists of: a hollow core; and a shell having a bilayer structure, which comprises the cyclic peptide.

14. The peptidesome according to claim 12, wherein a hydrophilic drug is captured in the core moiety and a hydrophobic drug is captured in the shell moiety so as to allow multiple drug release.

15. The peptidesome according to claim 12, wherein the peptidesome has an average diameter of 10-150 nm.

16. The peptidesome according to claim 12, wherein the peptidesome has an average shell thickness of 1-20 nm.

17. The peptidesome according to claim 12, wherein the cyclic peptide is a mixture of two cyclic peptides having different hydrophilic peptides (a).

18. The peptidesome according to claim 17, wherein the mixture of cyclic peptides is a mixture of a first cyclic peptide having a hydrophilic peptide selected from SEQ ID NOS 1-7 and a second cyclic peptide having a hydrophilic peptide selected from SEQ ID NOS 8-11.

19. The peptidesome according to claim 18, wherein the first cyclic peptide is represented by any of Chemical Formulas 1-3 and the second cyclic peptide is represented by Chemical Formula 4:

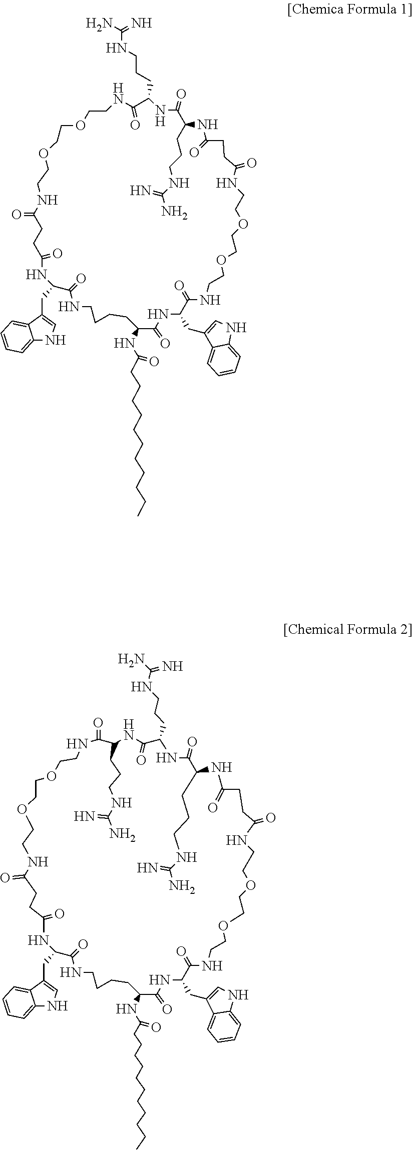

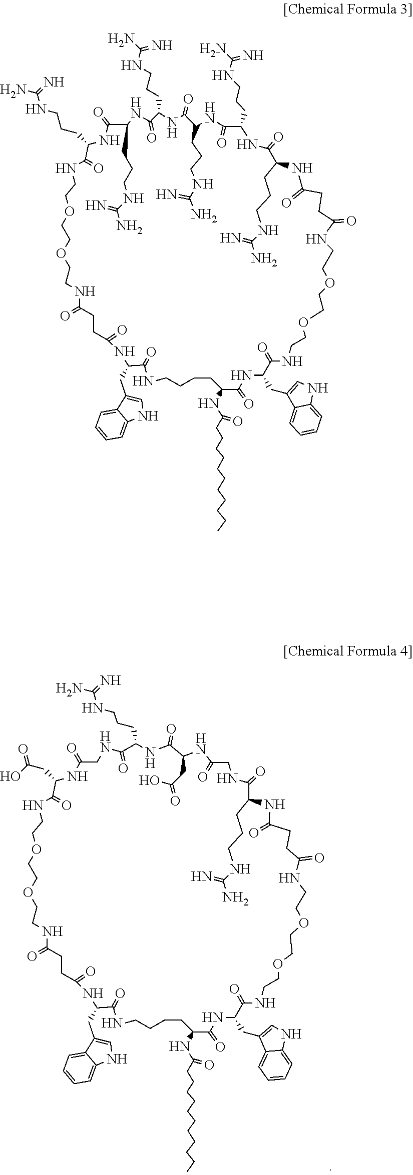

20. The peptidesome according to claim 18, wherein the mixture of cyclic peptides comprises 1-50 mol % of the first cyclic peptide and the second cyclic peptide as the balance.

21. The peptidesome according to claim 12, wherein the liquid is a solution comprising one or more solution selected from a group consisting of glucose, a polyol and distilled water.

22. The peptidesome according to claim 12, wherein the liquid is a solution comprising 1-10 wt % of glucose, 10-30 wt % of a polyol and distilled water as the balance.

23. The peptidesome according to claim 21, wherein the polyol is one or more polyol selected from a group consisting of ethylene glycol, propanediol, butanediol, pentanediol, hexanediol, glycerol and polyethylene glycol.

24. A pharmaceutical composition for preventing or treating cancer, comprising the peptidesome according to claim 12 and a drug encapsulated in the peptidesome.

25. The pharmaceutical composition according to claim 24, wherein the drug is any one selected from a hydrophilic drug, a hydrophobic drug and a mixture thereof.

26. The pharmaceutical composition according to claim 24, wherein a hydrophilic drug is encapsulated in a core of the peptidesome and a hydrophobic drug is encapsulated in a shell having a bilayer structure of the peptidesome.

27. The pharmaceutical composition according to claim 26, wherein the hydrophobic drug is any anticancer agent selected from doxorubicin, paclitaxel, cisplatin, sirolimus and etoposide or any photosensitizer selected from a phthalocyanine-based compound, a porphyrin-based compound, a fluorescein-based compound and a chlorin-based compound.

28. The pharmaceutical composition according to claim 24, wherein the peptidesome penetrates directly into cancer cells rather than through endosomes and primarily releases a hydrophobic drug, and then the peptidesome is disrupted by photodynamically generated reactive oxygen species and secondarily releases a hydrophilic drug comprised in a core.

29. The pharmaceutical composition according to claim 24, wherein the cancer is selected from a group consisting of lung cancer, stomach cancer, glioma, liver cancer, melanoma, kidney cancer, urothelial cancer, head and neck cancer, Merkel cell carcinoma, prostate cancer, blood cancer, breast cancer, mammary gland cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, nasopharyngeal cancer, thyroid cancer, bone cancer and a combination thereof.

30. A composition for diagnosing cancer, comprising the peptidesome according to claim 12 and a contrast agent.
